(19) United States
(12) Patent Application Publication
(10) Pub. No.: US 2007/0051362 A1
(43) Pub. Date: Mar. 8, 2007

(54) Multiple unit dose drug delivery system

(76) Inventors: Timothy R. Sullivan, Austin, TX (US); Jeffrey Nelson, Round Rock, TX (US)

Correspondence Address: VINSON & ELKINS, L.L.P., 1001 FANNIN STREET, 2300 FIRST CITY TOWER, HOUSTON, TX, 77002-6760, US

(21) Appl. No.: 11/114,251

(22) Filed: Apr. 25, 2005

Related U.S. Application Data
(60) Provisional application No. 60/564,734, filed on Apr. 23, 2004.

(51) Int. Cl. A61M 11/00; A61M 31/00

(52) U.S. Cl. 128/200.23; 604/500

(57) ABSTRACT

The present disclosure is directed to devices that administer single or multiple doses of one or more substances to the eye, nose, or ear of a user. The precise and repeatable dosing features of the presently disclosed devices overcome many of the disadvantages associated with known methods for dispensing substances to, for example, the eye of a user. The devices administer precise doses of a substance to a precise location from ampoules that may be single-dose or two-dose ampoules, which may be externally or internally pierced.

### STATEMENT REGARDING FEDERALLY SPONSORED RESEARCH OR DEVELOPMENT

Not applicable.

### REFERENCE TO A “Microfiche Appendix”

Not applicable.

### BACKGROUND OF THE INVENTION

1. Field of the Invention

The present disclosure relates to a device that administers single or multiple unit doses of a liquid, gel, or powder, or other substance containing an active ingredient to the eye, nose, or ear of a user.

2. Description of Related Art

While the development of pharmaceutical drugs is important for continued improvement of therapeutic alternatives, pharmaceutical drug delivery methods can also play a crucial role in making drugs readily available to patient populations. The easier a therapeutic drug is to administer, the more interested a potential patient will be in the drug and greater compliance with taking the drug will be achieved. For example, transdermal patch delivery of nitroglycerin more than tripled the nitroglycerin market, because it made the benefits of this drug conveniently available to patients. Other drug delivery systems that have increased the availability of pharmaceutical drugs to patients are lozenges, topical creams and gels, oral cancer drugs, sustained release medicines, liposomes, and medical device applications, to name but a few.

Despite the advances made in other areas for novel drug delivery systems, the ophthalmic industry has lagged behind in improving the administration of drugs to users. Eye drops have been used for over 100 years for front of the eye diseases, and are still the most widely used method for administering drugs to the eye. In fact, over 95% of all ophthalmic drugs are delivered through a traditional eye drop bottle delivery system. But because drops administered from an eye drop bottle are relatively large, the instinctive blink that is provoked by the arrival of the large drop severely limits the amount of or proportion of fluid that actually contacts the target area on the eye. For example, less than 10% of a 50 μl drop may deliver effective treatment for a patient's eye, with the remainder lost by drainage. The problem of drainage is further compounded by the natural limitations of the human eye to hold 10 μl to 12 μl before overflow occurs. This loss of expensive treatment fluids is wasteful, and leads to uncertainty about the effectiveness of a treatment. For chronic users of certain ophthalmic drugs, this problem of overflow can also cause allergic reactions to the eyelid or in some cases staining of the skin surrounding the eye. Thus, this method of delivery, while affording a measure of simplicity for the user, has a number of problems, including waste and cost arising from errors in drug administration; over or under medication arising from inexact administration of the drug; the need for preservatives in the drug to protect the efficacy of the drug once the dropper bottle is opened and exposed to air; eye irritation from exposure to preservatives required to maintain drug shelf life; loss of sterility or cross contamination of the drug; waste arising from discarding partially used bottles of the drug; accidental injury to the eye during administration; no easy means of tracking compliance to the prescribed use of the drug; and inadvertent use of expired drug supplies.

Ophthalmic drug delivery systems have been difficult to develop primarily because the eye has natural protective barriers, and is particularly sensitive to devices, implants and compounds that deliver drugs to the eye. Most, if not all, research by ophthalmic drug delivery systems companies, has been concentrated in efforts for diseases for the back of the eye, one of which is age-related macular degeneration (AMD). Within the past decade, there have been a limited number of new technologies developed that attempt to treat “front of the eye” disorders and diseases. These devices have been largely limited to single unit dose systems. The commercial success of these systems is limited because they do not meet the critical challenge of making drug administration to the eye simple, cost effective and convenient. There is a market need for an effective multiple unit dose delivery system for front of the eye ophthalmic drug administration, as evidenced by a study conducted by Beta Research Corporation, Syossett, N.Y., several years ago of a single unit dose administration using a first generation device to administer an ophthalmic drug to the front of the eye as an alternative to eye drops.

Another important consideration for the continued development of drug delivery systems is our aging population, and the increased care that people in this category need over time. For example, there are approximately 11.5 million people in nursing and assisted care centers in the U.S., and 59% need their medication administered by an assistant, taking up valuable resources, and depriving these people of their independence. Thus, there is a need for a comprehensive solution for certain patient populations, for example the elderly or those who are incapacitated, to self-administer pharmaceutical drugs in an easy and correct way. Some of the challenges facing institutional healthcare environments with respect to the administration of ophthalmic drugs to patients and residents include the time spent by caregivers administering eye drops to patients; potential liability as a result of accidental eye injuries which occur from faulty administration; increased cost due to waste; effective ophthalmic drug administration to uncooperative elderly and pediatric patients; cross contamination arising from using large institutional eye drop bottles; and the rising cost of drugs. Thus, an effective solution for addressing the shortcomings of using eye drop bottle delivery systems is needed.

### BRIEF SUMMARY OF THE INVENTION

The present disclosure is directed to devices that administer single or multiple doses of one or more substances, for example a liquid, powder, or gel, to a user, preferably to the eye, nose, or ear of the user. As used herein, the term “substance” includes but is not limited to an active-ingredient-containing substance wherein the active ingredient may be an active pharmaceutical ingredient (API), for example a pharmaceutical drug such as a prescription drug, generic drug, or over-the-counter pharmaceutical, neutraceutical or homeopathic product in an aqueous, gel, powder, solution, or suspension form. As used herein, an “active ingredient” is any component intended to furnish pharmacological activity or other direct effect in the diagnosis, cure, mitigation, treatment, or prevention of disease, or to affect the structure or any function of the body of humans or other animals. To achieve delivery of the substance, the substance can be atomized, aerosolized, or otherwise put into a particularized or droplet form, to be delivered to the eye, nose or ear.

The precise and repeatable dosing features of the presently disclosed devices overcome many of the disadvantages associated with known methods for dispensing substances to, for example, the eye of a user. In certain embodiments, the device makes the administration of the desired substance, for example an ophthalmic drug, simpler, faster, more convenient, safer, and less costly. In addition, in certain embodiments, the devices disclosed herein offer one or more of the following advantages: cost savings (reduces waste from over administration); improved efficacy from exact dosage administration; convenience and ease of use; improved patient compliance; improved safety; no cross contamination; reduces or eliminates the need for preservatives, thereby reducing the irritation and stinging the user would otherwise experience from the preservative; improved performance due to multi-unit dosing; and improved ability to meet the needs of elderly, incapacitated, and pediatric patients. In certain embodiments, this device also reduces potentially unpleasant side effects from the administration of certain drugs, and difficulties associated with eye dropper delivery systems.

For example, in the ophthalmic industry some eye drop units of liquid on the market are marketed as single-dose vials. These single-dose vials are manipulated and administered to the eye in the same manner as an eye drop bottle with all the same shortcomings. An important advantage of the presently disclosed device is that the ampoules used in the device to dispense a substance cannot be repeatedly used by the user. The one-time use nature of these ampoules eliminates the reuse problem common with other marketed eye drop units, and the risks associated with improper reuse of unit doses. Another advantage of the presently disclosed device is that it utilizes ampoules that maintain the sterility of the substance administered to the user until the moment of use. Since the sterile substance is not exposed to air until actual usage, loss of sterility is avoided. In addition, the mechanism of dispersal of the ampoules disclosed herein will prevent dispersion of the substance from any ampoules that are defective or have been damaged.

Another important advantage of the presently disclosed devices is that they will dispense precise amounts of the substance to a precise location in the eye, nose or ear, thereby reducing the risk of over or under medication. The more precise delivery system of the present disclosure also reduces waste from excessive or error prone delivery normally encountered with traditional eye dropper bottles, or other devices for delivery of drugs to the eye, nose or ear.

In preferred embodiments of a device designed for self-administration, the device has a clamshell, disk or cylindrical configuration. In preferred embodiments of a device designed for institutional administration, the device has a revolver configuration. The device preferably comprises a receptacle for receiving the Ampoule Cartridge Holder (“ACH”), which is designed to contain an assembly of ampoules which are either individual or interconnected by a web. Preferably, the ACH has a geometric tab or key or alternatively a flange that will allow it only to fit into the device one way, for example by using an indexing orientation key which mates to a key in the device. In another embodiment, the ACH is an integral component of the device. In yet another embodiment, the device comprises a substance release opening adjacent to the pierceable section of an ampoule. In still other embodiments, the device comprises a cylinder that comprises a linear belt feed or tube configuration of the ampoules, wherein the ampoules may or may not be interconnected.

In a preferred embodiment, the ACH is a disk, and in other preferred embodiments, the ACH is a tube or rectangular box. In certain preferred embodiments, the piercer is an integral part of the ampoule, while in other embodiments a compartment or ACH in the ampoule comprises the piercer. Preferably the substance is released or dispersed from the ampoule by compressing the ampoule with a piston, plunger, or roller, while simultaneously piercing the ampoule either internally or externally. In other embodiments, the ampoule further comprises a head space of air or gas, wherein compression of the ampoule provides the force required to activate the piercer, and the resulting expansion of the compressed air or gas assists in dispersing the substance out of the ampoule.

In some embodiments, a release button is an integral part of the device, and is operably connected to the spring loaded trigger such that pressing the release button activates the spring loaded trigger. Preferably the firing mechanism of the device consists of a piston, plunger, or roller powered by a hinge, spring, cam, or motorized drive, and is operably linked to a mechanical or electric power source. In other embodiments, the cam is spring driven or motor driven, for example by a battery.

The device can also further comprise a programmable microprocessor, preferably a Printed Circuit Board (PCB) or an Application Specific Integrated Circuit (ASIC) coupled to visual display interface, preferably a Liquid Crystal Display (LCD) or Light Emitting Diodes (LED), and audible signals which can be programmed to provide the user with prescription compliance notification and tracking, the operational status of the device, and the substance contained in the device. In other embodiments, the device comprises a magnified inspection window to inspect the ampoule cartridge in the device, so that the user may visually determine the type of substance loaded in the device, the number of remaining ampoules, and whether an ampoule has been administered by the device.

Preferably, the devices disclosed herein are used to administer one or more therapeutically effective substances to a user, for example the eye, nose, or ear of a user. In preferred embodiments, the device is used to administer one or more ophthalmic drugs to the eye of a user. In preferred embodiments, the device used for ophthalmic drug administration further comprises an eye cup that is preferably adapted to conform to the shape of the user's facial area surrounding the eye socket of the user. In preferred embodiments, the device comprises an eye cup storage space for a reusable eye cup. In other preferred embodiments, the eye cup is an Integrated Ampoule Ophthalmic Dispenser (IAOD), and the device may optionally comprise a spring loaded IAOD ejection mechanism. In preferred embodiments, the caregiver is not required to physically touch the IAOD either before or after the administration process, reducing cross-contamination risks.

### BRIEF DESCRIPTION OF THE SEVERAL VIEWS OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 is a cross-sectional illustration of a preferred embodiment of an internally pierced ampoule.

FIG. 2 is an illustration of a preferred embodiment of a cutaway tip ampoule.

FIG. 3 is an illustration of alternate preferred embodiments of cutaway tip ampoules.

FIG. 4 is an illustration of a preferred embodiment of an externally pierced ampoule.

FIG. 5 is an illustration of a preferred embodiment of a burst ampoule.

FIG. 6 is an illustration of a preferred embodiment of a self-piercing ampoule.

FIG. 7 is an illustration of a preferred embodiment of a drug delivery device in which a hand actuated paddle like device is used to administer a drug. The device is shown in the storage configuration.

FIG. 8 is an illustration of the embodiment of FIG. 7 in a ready to fire configuration.

FIG. 9 is an exploded view of the embodiment of FIG. 7.

FIG. 10 is cross-sectional views of a preferred embodiment of a drug delivery device that is cocked by a lever on the bottom of the device, wherein the device is shown in cartridge replacement, ready to be fired, fired, and storage states.

FIG. 11 is an illustration of a preferred embodiment in which an external dial device is used to cock the firing mechanism.

FIG. 12 is a illustration of a preferred embodiment of a drug delivery device in which two wing-like members are manipulated to cock the device. The device is shown in the open position.

FIG. 13 is an illustration of the embodiment of FIG. 12 in the closed, storage position.

FIG. 14 is an illustration of a preferred embodiment of a device in which the cocking mechanism and other functions are powered by a battery. The device is shown in the open and stored configurations.

FIG. 15 is an exploded view of the embodiment of FIG. 14.

FIG. 16 is a flow chart of an embodiment of electronic control of a preferred drug delivery device.

FIG. 17 is cross-sectional views of a preferred embodiment of a drug delivery device that uses a roller to compress a burst ampoule.

FIG. 18 is cross-sectional views of a preferred embodiment of a drug delivery device that is cylindrical, wherein the device is shown in storage, ready to be fired, fired, and ejecting ampoule states.

FIG. 19 is an illustration of a preferred embodiment of a drug delivery device preferably for use by a care giver or institutional user.

FIG. 20 is an illustration of a package of integrated ampoule eyecups.

FIG. 21 is a cross-sectional illustration of a preferred embodiment of a drug delivery device preferably for use by a care giver or institutional user.

FIG. 22 is an illustration of a preferred embodiment of a drug delivery device for nasal or otic administration.

FIG. 23 is an illustration of a preferred embodiment of a drug delivery device for nasal or otic administration.

### DETAILED DESCRIPTION OF THE INVENTION

The present disclosure is directed to devices able to dispense single or multiple doses of one or more substances that preferably contain an active ingredient (such as a pharmaceutical drug), to a user. The devices can be modified to dispense the substance to the eye, nose, or ear of a user. As used herein, the term “user” is interchangeable with the terms “subject” or “patient,” and refers to a mammal, preferably a human, but also can refer to animals, for example, cats, dogs, mice, cows, horses, pigs, and the like. Preferably the devices incorporate an ergonomic design that makes the devices easy to operate and reduces the time needed for administering substances. In preferred embodiments, the devices are portable hand-held devices that utilize disposable ampoules containing the substance to be administered to the user, as well as an eyecup. The devices can be configured either for self-administration, or for use by a caregiver, such as medical and health care professionals, for example, in an institutional setting such as a hospital, clinic, nursing home, assisted living environment, physicians office, and pediatric center. In certain preferred embodiments, the devices are used for ophthalmic drug delivery applications, such as for the treatment of dry eye, allergies, glaucoma, cataracts, or other chronic eye problems or diseases, as well as the administration of anti-infectives such as antibiotics or bacteriostatic compounds, anti-inflammatories, or biologics.

One disadvantage of using eye drop bottles to dispense liquids to the eye is that often times too much liquid is administered to the eye, or the dispensed droplet misses the eye, resulting in waste of the liquid, as well as potentially resulting in over or under dosing of the medication. For example, with a conventional eye-dropper, the smallest droplet that will free fall from the tip of the dropper is approximately 35 μl of liquid due to the effect of surface tension between the liquid, the tip of the dropper, and the liquid remaining inside the tip. Considering the maximum volume of liquid that the eye can receive is 10 μl, a significant portion of the administered liquid is wasted. The presently disclosed devices overcome these drawbacks by allowing for the administration of smaller volumes of liquid to, for example, the eye of a user. Preferably, the liquid dispensed by the devices is atomized, and discharged as a coherent stream of droplets, for example into the user's eye. Alternatively, the liquid can be dispensed as a fine mist into the user's nose. Another advantage of the presently disclosed devices is that the eyecup reduces the user's eye blink rate, which means the patient is less likely to blink during administration. In addition, since the devices disclosed herein preferably atomize the liquid, the user is less likely to blink during administration, which facilitates administration of the liquid to the user.

Another disadvantage of conventional eye-droppers that is overcome by the presently disclosed devices is that the amount of substance dispensed with an eye-dropper will depend on the amount of force the user applies to the eyedropper bottle, which presents an uncontrolled variable into the administration of a substance to the eye. In contrast, the devices of the present disclosure dispense a discrete volume of substance to a precise destination with each administration, independent of the coordination of the user. The ease of administration is particularly important for elderly or incapacitated users, who typically find it difficult to apply eye drops because of a physical infirmity, such as arthritis, or other disabling conditions. Thus, certain embodiments of the presently disclosed devices allow a user to dispense a required dosage of substance accurately and easily. Another advantage is that since gravity is not required for dispensing substance with these devices, as it is with eye-droppers, the devices can be operated from a wide range of physical orientations, for example in an upright, horizontal, vertical, or downward position. This minimizes the need for the user to tilt back the head during administration and reduces risks associated with loss of balance or neck injury.

In preferred embodiments, the volume of droplets or particles dispensed from the devices to the eye is from about 1 μl to about 25 μl, more preferably from about 2, 3, 4, 5, 6, 7, 8, 9, 10, 11, 12, 13, 14, 15, 16, 17, 18, 19, 20, 21, 22, 23, or about 24 μl. The volume and size of droplets or particles released by a device can be adjusted to maximize the therapeutic benefit of the dispersed substance. The volume of substance dispensed depends on the size of the compartment containing the substance, the ampoule, the piercer, and other variables in the construction of the devices, as well as characteristics of the substance dispersed, which are well understood by those skilled in the art. These variables can be appropriately dimensioned to achieve dispersal of a desired volume or droplet size of liquid or particle size of substance to the user. Preferably, the droplets or particles dispersed from the devices disclosed herein are large enough such that they do not form an inhalable spray, for example droplets greater than about 20 μm in diameter. One of skill in the art understands that residual liquid or other substance after dispersal is taken into account when formulating the appropriate parameters for dispersing the desired dosage volume.

An advantage of the device and ampoule designs set forth herein is that the sterility of the administered substance is maintained until the moment of use. Maintaining sterility until the moment of use minimizes or eliminates the need to use preservatives or bacteriostatic compounds in the substances administered, without risking contamination. In addition, if the ampoule is damaged, or is otherwise defective, the devices do not administer the substance, which may no longer be sterile. For example, if an ampoule is defective in the area of the pierceable section, or develops a leak, the devices will not dispense the substance properly because sufficient pressure will not be generated in the ampoule to effectively release the substance.

In preferred embodiments, the substance dispensed from the devices disclosed herein is an active pharmaceutical ingredient (API), including but not limited to the following therapeutic compounds: anti-glaucoma/IOP (intra-ocular pressure) lowering compounds (e.g., β-adrenoceptor antagonists, such as carteolol, cetamolol, betaxolol, levobunolol, metipranolol, timolol; miotics, such as pilocarpine, carbachol, physostigmine; sympathomimetics, such as adrenaline, dipivefrine; carbonic anhydrase inhibitors, such as acetazolamide, dorzolamide; and prostaglandins, such as PGF-2 alpha); anti-microbial compounds, including anti-bacterials and anti-fungals, e.g., chloramphenicol, chlortetracycline, ciprofloxacin, framycetin, fusidic acid, gentamicin, neomycin, norfloxacin, ofloxacin, polymyxin, propamidine, tetracycline, tobramycin, quinolines; anti-viral compounds, e.g., acyclovir, cidofovir, idoxuridine, interferons; aldose reductase inhibitors, e.g., tolrestat; anti-inflammatory and/or anti-allergy compounds, e.g., steroidal compounds such as betamethasone, clobetasone, dexamethasone, fluorometholone, hydrocortisone, prednisolone, and non-steroidal compounds such as antazoline, bromfenac, diclofenac, indomethacin, lodoxamide, saprofen, sodium cromoglycate; artificial tear/dry eye therapies, comfort drops, irrigation fluids, e.g., physiological saline, water, or oils; all optionally containing polymeric compounds such as acetylcysteine, hydroxyethylcellulose, hydroxymellose, hyaluronic acid, polyvinyl alcohol, polyacrylic acid derivatives; diagnostics, e.g., fluorescein, rose bengal; local anesthetics, e.g., amethocaine, lignocaine, oxbuprocaine, proxymetacaine; compounds that assist healing of corneal surface defects, e.g., cyclosporine, diclofenac, urogastrone and growth factors such as epidermal growth factor; mydriatics and cycloplegics, e.g., atropine, cyclopentolate, homatropine, hysocine, tropicamide; compounds for the treatment of pterygium, such as mitomycin C, collagenase inhibitors (e.g., batimastat); compounds for the treatment of macular degeneration and/or diabetic retinopathy and/or cataract prevention; and compounds for systemic effects following absorption into the bloodstream after ocular, intranasal, or otic administration, e.g., chemical drugs, proteins and peptides such as pain medication for migraine or chronic pain management, vaccines, insulin, histamines, corticosteroids decongestants, and hormones.

In other preferred embodiments, the substance is particularly well suited for intranasal delivery, including but not limited to FluMist (Mediimmune), Imitrex (Glaxo), Migranal (Xcel), Miacalcin (Novartis), Nascobal Gel (Nastech/Questcor), Nicotrol (Pfizer), Stadol NS (Bristol-Myers-Squibb), Stimate (Aventis Behringer), Synarel (Pfizer), Zomig (AstraZeneca), Apomorphine (Britannia Pharm), Apomorphine (Nastech), Emitasol (Questor), Fentanyl (West Pharm), FluINsure (ID Biomedical), Fortical (Unigene), Hypnostat (Questcor), Insulin (Bentley Lab), Interferons (Nastech), Ketamine (IDDS), Leuprolide (West), Migrastat (Questor), Morphine (West), Morphine Gluconate (Nastech), Nascobal Spray (Questcor), Somatropin (Nastech), Peptide YY 3-36 (Nastech), PH948 (Pheriin), PH80 (Organon/Pherin), Triptan (Nastech), and Vaccines (West). In still other preferred embodiments, the substance is a vaccine, for example a vaccine to dipthteria, tetanus, acellular pertussis, Influenza, Herpes Simplex, Hepatitis A, Hepatitis B, Hepatitis C, Measles, Mumps, Rubella, Pneumoccal conjugate, Polio, Anthrax, Rabies, Typhoid, Yellow fever, and Attenuvax (Merck).

The active-ingredient-containing substances administered by the devices disclosed herein may be the free acid or free base form of the active-ingredient, or alternatively a salt of the active-ingredient. In addition, the devices disclosed herein may be used to treat a patient with one or more active-ingredient-containing substances. The active-ingredient-containing substances are preferably formulated as aqueous solutions, gels, powders, solutions, or suspensions, and these formulations may optionally contain other formulation excipients, including but not limited to thickening agents such as gels, mucoadhesives and polymers, stabilizers, anti-oxidants, preservatives, and/or pH/tonicity adjusters.

Ampoules

The devices disclosed herein are able to dispense single or multiple doses of one or more substances to a user by utilizing ampoules. As used herein, the term “ampoule” is interchangeable with the terms “bottle,” “vial,” “unit-dose vial,” or “container.” In preferred embodiments, an ampoule contains a single-unit dose of a substance, or a two-unit dose of one substance or two different substances, in one or more compartments of the ampoule. Alternatively, an ampoule may administer three or more substances from one or more compartments in an ampoule. During manufacture, the ampoules can be singulated, i.e., individually broken apart and individually loaded into, for example, an ampoule cartridge or a device. Alternatively, the ampoules can be interconnected, for example through a connected webbing. The ampoules can be manufactured as a strip of ampoules, which then may be manipulated into different forms for administration, such as a circle, ring, or tube. In other preferred embodiments, the ampoules themselves, or the Ampoule Cartridge Holder, adhere to a numbering, color coding, icon system coding, or Braille system for assisting the user in administration of the ampoules, and may also include a bar code or Radio Frequency Identification Device (RFID).

In general, the ampoules disclosed herein are single unit dose, sterile containers used to hold and dispense a wide range of substances to the eye, nose, or ear of a user. Preferred ophthalmic applications include using the ampoules to administer chemical drugs to treat glaucoma, allergies, or dry eye, or may be used to administer anti-infectives or anti-inflammatory drugs. Preferred intranasal drugs include but are not limited to chemical drugs for migraines, pain management, or allergies, or intranasal ampoules are designed to administer anti-infectives, vaccines, or insulin. Preferred otic drugs include but are not limited to anti-infectives and anti-inflammatory drugs. It is also understood that additional drugs known to those of skill in the art, as well as drugs yet to be discovered, may be administered to users by utilizing the presently disclosed ampoules and devices.

Preferably, ampoules utilized in the presently disclosed devices are small, ranging from about 0.5 cm to about 2 cm in diameter, more preferably from about 1 cm to about 1.5 cm in diameter. As a preferred embodiment, single-unit dose opthalmic ampoules range in size (interior volume) from 25 μl to 100 μl. Preferably, two-unit dose ophthalmic ampoules range in size (interior volume) from 50 μl to 120 μl. Preferably, single-unit dose intra-nasal ampoules range in size (interior volume) from 50 μl to 250 μl. Preferably, two-unit dose intra-nasal ampoules range in size (interior volume) from 100 μl to 500 μl. Preferably, otic ampoules will have interior volume capacities that fall within ranges similar to intranasal ampoules. It is understood that the volumes described in this paragraph are based on the volumes necessary to deliver an effective dose to the appropriate structure of the subject, and are primarily based on the treatment of human subjects. The optimal dosage of administered of a particular substance to a subject will be determined by methods known in the art and may vary depending on such factors as the subject's age, weight, height, sex, general medical/clinical condition, previous medical history, disease progression, formulation, concomitant therapies being administered, observed response of the subject, and the like. Ampoules for delivery to agricultural or domestic animals may also vary in size and volume appropriately.

It is also understood that when citing a range, such as “from 25 μl to 100 μl” the range of volumes is intended to include the end points, 25 and 100 μl in this example, and also is intended to include any volume within the range either in integer units or in fractions of integers. For example, the range “from 25 μl to 100 μl” would also include 26, 27, 28, 29, 30, etc. in unit increments up to 99 μl, as well as any fraction of the intermediate integers such as 25.1, 25.2 etc. It is also understood that the endpoints are not intended to be absolute and that a volume that falls within 10% of the end point would be considered to be within the range, for example, from 22 to 25 μl, or from 100 to 110 μl would reasonably be included within the range from 25 μl to 100 μl. This range is used as an example only, and the same description would apply to all volumes of ampoules described herein, as well as all ranges for various parameters disclosed herein.

In preferred embodiments, the ampoules contain a substance, for example an active-ingredient-containing substance, or a combination of both a substance and sterile air or other inert gas or vacuum depending upon the type of substance packaged in the ampoule, concentration of the substance, active ingredient bioavailability requirements, and the ampoule design utilized. In preferred embodiments the emitted dose efficiency (“EDE”) of an ampoule ranges from about 50% to about 90% depending upon the ampoule design utilized and fill volume ratio. The EDE is the ratio of the volume of substance actually delivered to the destination during the administration and the volume of substance contained in the ampoule.

It is an aspect of the present disclosure that the ampoules include an interior compartment, or drug compartment, that contains a substance to be administered, which is in fluid communication with, or is adjacent to, a pierceable section of wall of the ampoule. This wall may be an interior or exterior wall. Various ampoule designs and piercing embodiments are disclosed herein in which the section of the exterior wall of the ampoule may be pierced or opened by the appropriate device to release the substance in the ampoule. In certain embodiments, the compartment of the ampoule includes an internal piercer, in other embodiments the ampoule is opened by an external piercer, in still other embodiments the ampoule is opened by a blade that is part of the dispensing device, and in still other embodiments the ampoule is compressed by a roller designed to burst the ampoule in a pre-defined location of the exterior wall. All embodiments of the ampoules and devices preferably include a mechanism to automatically pierce or open the ampoule's compartment and dispense the substance contained therein to the subject upon firing the device (e.g., see U.S. Pat. No. 5,411,175, incorporated herein by reference). In certain embodiments, a piercer may either move toward and pierce the pierceable section of the compartment to allow dispersion of the substance, or the pierceable section may move toward and be pierced by the piercer. Alternatively, a blade piercer may move at or near a perpendicular to the central axis of an ampoule to facilitate release of the substance. Alternatively a roller piercer may compress the ampoule from above, below, or from opposite directions by moving along the length of the ampoule to cause the ampoule to burst open in a specific and pre-defined manner.

The piercers are preferably made from conventional materials, such as plastics, plastic laminates, plastic metal laminates, or metal, and are preferably constructed such that applying the appropriate pressure, for example by the device firing mechanism compressing the ampoule, causes the piercer to breach the pierceable section of the ampoule, thereby releasing the contents of the ampoule in a controlled manner. The internal piercer may be located in the same compartment as the substance, adjacent to the compartment, or external to the compartment. The ampoule can contain more than one internal piercer, and/or each piercer in an ampoule may contain one or more points that contact the pierceable section. For example, the ampoule may be designed such that the pierceable section is pierced by more than one piercer, or a piercer with more than one piercing point. Multiple piercing points may be used to increase the delivery rate of the substance in the ampoule. The piercer may be manufactured as an integral part of the ampoule, or independently of the ampoule. Preferably the substance in the compartment of the ampoule is sterile, and the sterility is maintained until the moment of administration. In some embodiments the piercer is hollow. In other embodiments the piercer is open or closed at the end distal to the pierceable section.

The ampoules disclosed herein are designed to “fire” or “burst” under force impact striking and compressing the ampoule from the rear or alternatively from one or both sides of the ampoule, depending upon the device fire mechanism design. The force required to effectively fire an ampoule is preferably about 2 to 8 pounds, although this is balanced with other design requirements associated with ensuring ampoule integrity, shelf life, vapor pressure performance, and the ampoule manufacturing process limitations. Complete discharge of the substance out of the ampoule and into the eye preferably occurs in approximately 200 to 300 milliseconds from the point of impact of the compressive force.

Preferably, the ampoule spray patterns are consistent from ampoule to ampoule and deliver the substance into the lower quadrant (i.e. the cul-de-sac of the eye). In preferred embodiments, the substance is driven out of the ampoule with sufficient linear energy to overcome the effects of gravity, i.e., the user is able to consistently deliver the substance into the eye reliably and safely when the user's head and eye orientation is perpendicular to the force/direction of gravity. Preferably, the substance is delivered in individual droplet sizes ranging from 2 μl to 10 μl, or alternatively 100 micron to 400-micron diameter droplets. Linear distance to be traversed by the substance from the ampoule to the surface of the eye is preferably between about 10 mm and 35 mm. The primary design considerations of ampoules and devices disclosed herein are consistency of the delivered dose to the targeted destination in the eye, nose or ear, droplet size, and force level that is comfortable, safe and efficacious for the user, as well as delivery precision to the targeted destination.

In certain embodiments of the present disclosure, a nozzle or spray head is positioned directly adjacent to the area of the ampoule from which the substance is released. The nozzle or spray head may be attached to the device or alternatively to the ampoule or cartridge. For example, the nozzle or spray head may fit over the pierceable section of the ampoule to provide an orifice through which the substance in the ampoule can be released. The nozzle or spray head may optionally direct the flow of the substance after it is released from the ampoule. In another embodiment, a device has a receptacle for receiving the nozzle or spray head attached to the cartridge, such that the nozzle or spray head is aligned with the receptacle to dispense the substance. In still other embodiments, the ampoule has a removable external cover or seal overlaying the outer area of the pierceable section, which may be removed just prior to the substance in the ampoule being discharged. The seal may be, for example, a separate peelable layer.

A preferred method of manufacturing ampoules is the process of Form Fill Seal (FFS), or alternatively Blow Fill Seal (BFS), which incorporate an aseptic sterilization process to produce sterile ampoules. For example, BFS, which is a process well known to those of skill in the art, is a specialized packaging technology using in-line forming and sealing of a polymeric material to a container of choice. BFS machines incorporate a polymer granule storage and feeding system, a rotating screw extruder with parison head, a sterile air-filling chamber, mold halves to form and close the container, and downstream equipment including for example leak-detection systems. Pharmaceutical BFS systems utilize an aseptic manufacturing process to produce sterile containers and drug products. Polymer granules are fed via vacuum tubing system into a hopper of the BFS extruder, where they are heated to a melt (160° to 170°). The homogeneous polymer melt is formed via a circular orifice into a plastic parison, which does not collapse because of a stream of sterile filtered air. Next, the lower part of the divided mould halves close to seal the bottom of the open parison and the parison wall is blown and/or sucked to the cooled mold walls to form the lower part of the container. Filling needles draw the stipulated volume of substance into the container and, after withdrawal of the filling needles, the upper part of the mold closes to form and seal the upper part of the container.

An alternative sterilization process is terminal sterilization using techniques such as gamma ray irradiation to produce sterile, preferably preservative-free drug packages. As a preferred embodiment, the ampoules are constructed of low density polyethylene (LDPE) or polypropylene, or high density polyethylene (HDPE), or other plastics, polymers, or exotic resins. More preferably, the ampoules are constructed of materials approved by the FDA for use in pharmaceutical packaging, which also preferably conform to FFS or BFS process specifications. As a preferred embodiment, the ampoules consist of an external container shaped as a cylinder, tube, teardrop, chevron, cone, rhombus, trapezoid, sphere, or partial sphere. Preferred ampoules may contain none, one, two, or more bellows, or alternatively may have a container shaped to aid in compressing or piercing the ampoules. Preferred ampoules have a single internal compartment or alternatively may contain one or more separate internal compartments adjacent to the compartment containing the substance. Preferably ampoule wall thickness ranges from about 0.05 mm to 2 mm. As a preferred embodiment, the ampoules are constructed from LDPE with a wall thickness of about 0.5 mm. In certain embodiments, the thickness of the ampoule wall varies in certain portions of the ampoule to aid in compression and/or piercing.

In certain preferred embodiments, ampoules are filled using a BFS process in the following ranges: a) single-unit dose ophthalmic ampoules contain a drug dose of 15 to 60 μl, b) two-unit dose ophthalmic ampoules contain a drug dose of 40 μl to 90 μl, c) single-unit dose intranasal ampoules contain a dose of 40 μl to 200 μl, and d) two-unit dose intranasal ampoules contain a dose of 60 μl to 400 μl. Preferably, otic ampoules have dose amounts that fall within ranges similar to intranasal ampoules. A preferred use of the two-unit dose ampoules is to fire two individual doses of a drug in sequence (e.g., right eye, then left eye of a single subject).

Ampoule safety design considerations include the capability to color code ampoules or Ampoule Cartridge Holders as an aid to identification. Ampoule design may include consideration for maintaining an acceptable level of vapor pressure migration that may affect the substance quality or efficacy over time. In preferred embodiments, shelf life of the ampoule and contained substance is targeted at a minimum of 12 months and preferably 24 months from date of manufacture.

Preferably the devices and ampoules incorporate one of the following mechanisms for piercing the ampoule and producing a coherent stream of droplets or spray plume for delivery of the substance in the ampoule to an eye, nose or ear: (a) internally pierced ampoules; (b) cutaway tip ampoules; (c) externally pierced ampoules; (d) burst ampoules; and (e) self-piercing ampoules.

Internally Pierced Ampoules

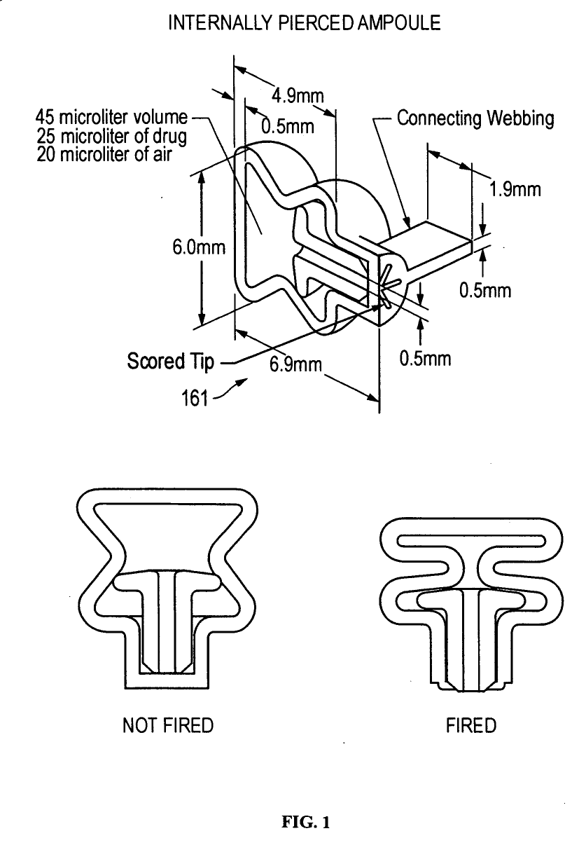

An example of an internally pierced ampoule is shown in FIG. 1. The internal piercer 161 can take different shapes, including but not limited to a funnel design in which the drug is deposited, or a disc shape design. Preferably, the ampoule is scored or has a frangible wall on the exterior container at the penetration point of the piercer to reduce the penetration force required to pierce the pierceable section. This feature will also minimize the release of any foreign materials from the ampoule wall during administration. The wall thickness of the ampoule may be varied to aid in compression of the ampoule or penetration of the piercer. In preferred embodiments, the internal piercer is constructed of styrene, polystyrene, or other pharmaceutical grade FDA approved materials of sufficient hardness to penetrate the ampoule wall.

In preferred embodiments, the internal piercer includes a hollow tube or channel (the delivery channel) through which the substance flows as the ampoule is compressed and pierced. The tip of the piercer preferably has an angled edge to aid in penetration of the ampoule container. The inside diameter of the piercer tube can range from about 0.015 inches to about 0.05 inches, but in certain preferred embodiments is about 0.025 inches. The internal diameter, shape, or surface texture of the delivery channel near and at the exit point may contain a nozzle or may be varied to form the optimum droplet size and spray plume geometry of the sample as it exits the ampoule. Internally pierced ampoules may be single-dose or two-dose units.

Internally pierced ampoules may be produced during the BFS manufacturing process in a linear strip. In a preferred embodiment, individual ampoules are connected via a connective webbing that is made of the same material as the ampoules, and that is formed during the BFS manufacturing process. The ampoules can be singulated, i.e., individually broken out of the strip form and placed into an Ampoule Cartridge Holder, or preferably the entire strip of ampoules is formed into an Ampoule Cartridge Holder, or alternatively formed into a series of ampoules, for example in the shape of a ring, circle, or tube.

Cutaway Tip Ampoules

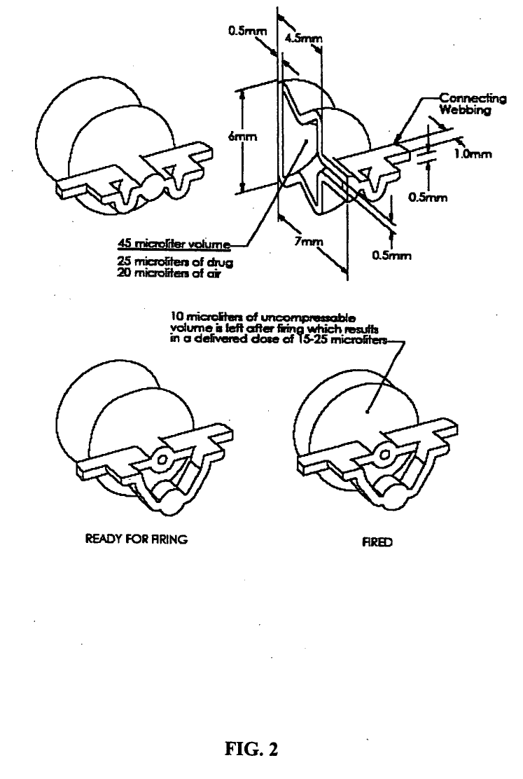

Cutaway tip ampoules preferably utilize a novel cutaway tip, as shown in FIG. 2, to create a precise opening in the ampoule to facilitate dispensing of the substance in a coherent spray. In certain embodiments the cutaway ampoule includes a flexible hinge on the cutaway tip of the ampoule. During use, a cutter blade edge in the device cuts the tip of the ampoule during the firing sequence. The severed tip is attached by two plastic spring hinges on either side of the tip. The cutter blade pushes and holds the severed tip down to open the dispensing tube. When the ampoule is compressed the liquid is forced out through the opening. Preferably, the severed tip is returned to the original position upon completion of the firing sequence to seal the ampoule, thereby preventing leakage of any remaining liquid.

Severing the tip of the ampoule can occur anywhere from the connection point of the tip to the main ampoule container to within about 0.5 mm of the end of tip. In preferred configurations, the tip is severed about 1.5 mm from the end of the tip leaving about a 1.0 mm dispensing channel on the ampoule. It is understood, however, that overall dimensions may vary with the volume capacity of the ampoule.

Alternative configurations for the cutaway tip include a single pivoting hinge and a cutaway tip that is completely severed from the ampoule and captured in a small compartment in the ampoule cartridge or the device. In other embodiments, the severed cutaway tip is ejected from the device.

Cutaway ampoules have the potential to drip or release a small droplet of the substance, particularly if it is liquid, at the time they are cut open. This can result in small amounts of substance collecting in the dispensing channel as a result of capillary action either during storage or at the point of piercing. In certain embodiments, surface tension of the liquid in the ampoule container will reduce the potential for inadvertent leakage. This natural dynamic can be enhanced through appropriate design of the internal structure of the ampoule and the dispensing channel.

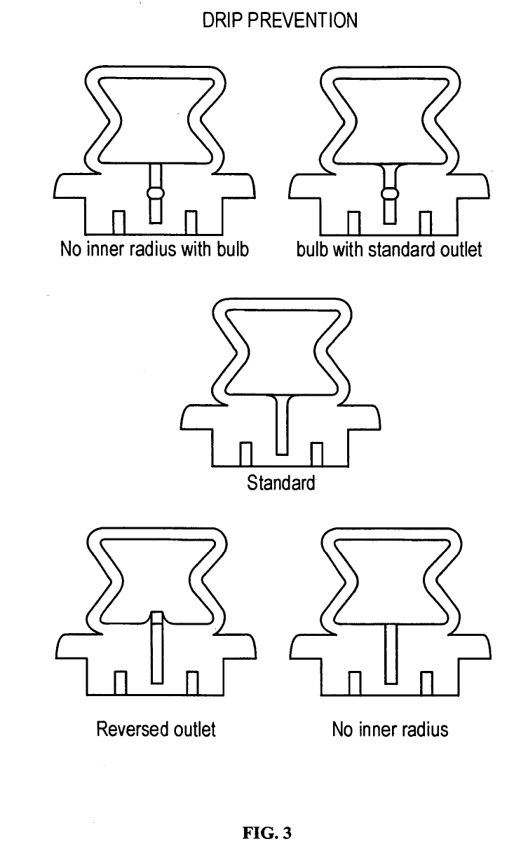

Various techniques and internal ampoule designs have been developed to reduce or prevent inadvertent release of substance during the piercing process on ampoules. These design features entail modifying the dispensing channel, or modifying the intersection of the dispensing channel where it intersects the primary chamber of the ampoule container as shown in FIG. 3. For example, an ampoule can be designed for providing a substance to be dispensed, the ampoule comprising a compartment for holding the substance, the compartment having the substance disposed therein and comprising a dispensing channel with no inner radius, no inner radius with a contouring wall (e.g., a bulb), a contouring wall with a standard outlet, or a reversed outlet, wherein the design of the dispensing channel reduces or prevents inadvertent release of the substance during the piercing process. All of these design configurations help inhibit the collection of the substance, particularly in liquid form (for example by capillary action), in the dispensing channel during, for example, storage and handling of the device, or after the ampoule is pierced but prior to compression of the ampoule to release the substance. It should be noted that these design techniques can be used individually or in combination. Additionally, one can reduce inadvertent drip through manipulation of the ampoule during the firing sequence. As an example, the device can be configured to stretch the ampoule during the firing sequence just prior to the piercing and compression step. This action draws a small volume of air into the device through the dispensing channel as it is pierced and prevents liquid from leaking.

Externally Pierced Ampoules

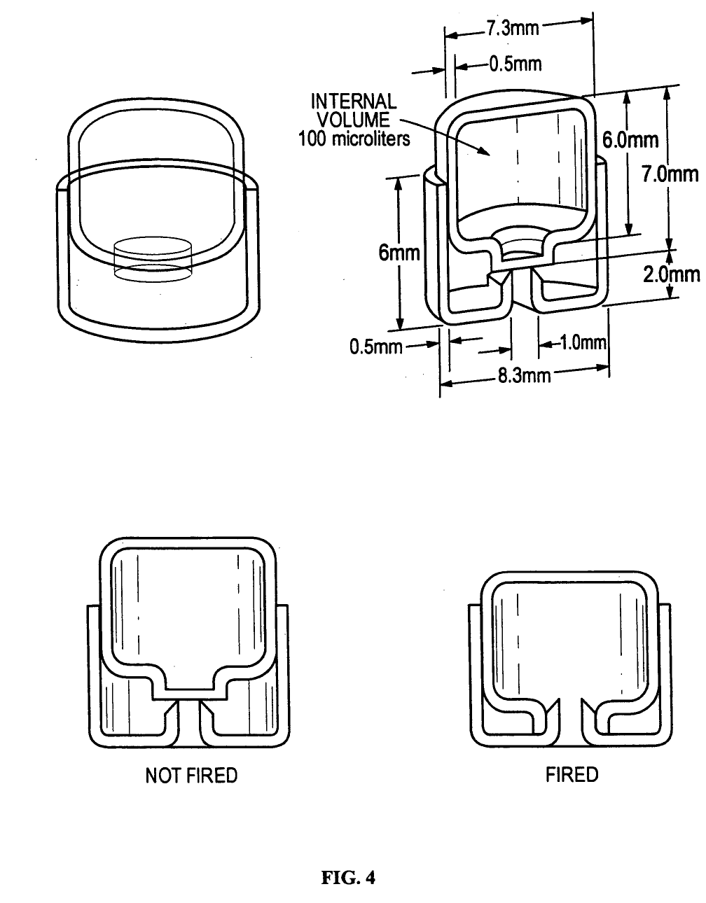

Externally pierced ampoules utilize an external piercer that contains a hollow delivery channel to pierce and fire the ampoule. In preferred embodiments, the piercer is integrated into the ampoule and each ampoule has its own single-use piercer to reduce potential for contamination. Alternatively, the external piercer is integrated into the Ampoule Cartridge Holder. In certain embodiments, as the device compresses the ampoule it moves in two dimensions: (a) the entire ampoule moves forward in the ACH towards and onto the piercer; and (b) the ampoule is compressed from the back as the plunger or piston in the device puts pressure on the ampoule forcing the liquid out the hollow core of the delivery channel. An alternative configuration for the externally pierced ampoule is illustrated in FIG. 4. This design contemplates an individual piercer constructed of styrene, polystyrene or similar pharmaceutical grade plastic which attaches directly to the individual ampoule and is then loaded into the ampoule cartridge.

Externally pierced ampoules may or may not be linked via a connective webbing at the time they are loaded into the ampoule cartridge, since they may be required to move as they are fired. If the ampoules move during administration by the device, they are preferably singulated during the manufacturing process and individually loaded into the ampoule cartridge in a post production process. Overall dimensions of externally pierced ampoules will vary with the volume capacity of the ampoule.

Burst Ampoules

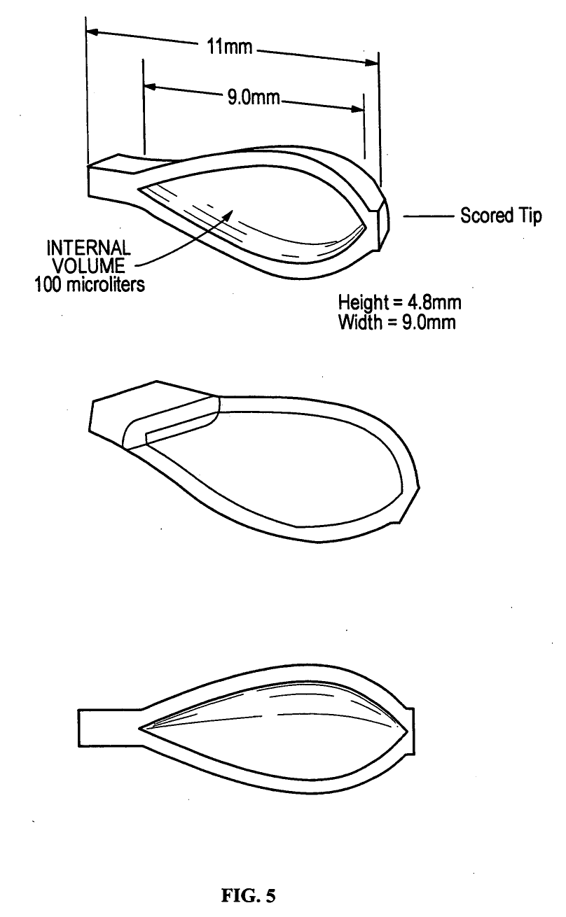

Burst ampoules are designed to burst in a pre-defined location when compressed. The design as shown in FIG. 5 demonstrates a compression technique which squeezes the ampoule from the rear to the front in a progressive manner.. Preferably, a frangible or scored surface at a predetermined location of the ampoule, for example at the front of the ampoule, is the defined burst point. The ampoule wall is thinner at this point to reduce the force required to burst the ampoule and to insure that release of the substance in the burst ampoule occurs at the intended location. Overall dimensions will vary with the volume capacity of the ampoule. Wherein many of the preferred embodiments utilize a shaft to compress the ampoule from the bottom, any of these mechanisms can be modified to include a pair of rollers to engage the ampoule from the sides and compress until the ampoule bursts and releases the substance it contains.

Self-Piercing Ampoules

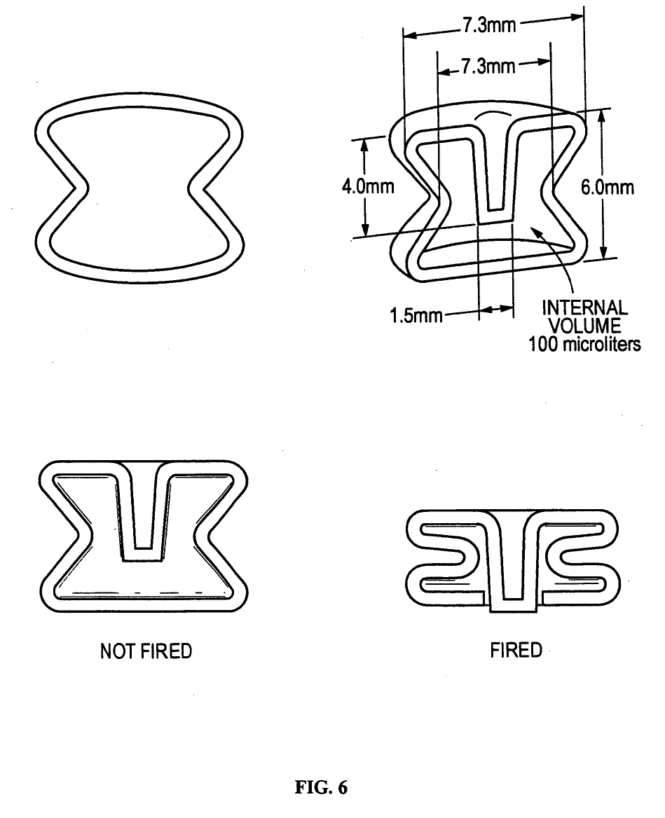

Self-piercing ampoules are designed such that the ampoule shell is modified to incorporate an internal piercer which serves to pierce the ampoule when, for example, a plunger with a finger rod is inserted into the self-piercer to reinforce it and drive it through the front of the ampoule while compressing the ampoule. Self-piercing ampoules, as shown in FIG. 6, preferably have a delivery channel that is a series of small channels formed on the surface of the piercer. Liquid is forced through into the channels and flows out of the ampoule riding on the surface of the self-piercer. The finger rod on the plungers serves to reinforce the internal self-piercer to provide the support necessary for the self-piercer to penetrate the pierceable section of the ampoule. The finger rod provides sufficient strength and support to enable a piercer constructed of a like material as the ampoule container to penetrate the wall of the ampoule. Preferably, the ampoule has a frangible or scored exterior surface at the penetration point of the piercer to reduce the force required to pierce the ampoule and reduce the potential for release of foreign material off the ampoule during the piercing process. Overall dimensions will vary with the volume capacity of the ampoule.

Ampoule Cartridges

Preferably, ampoules are produced during the BFS manufacturing process in a linear strip with a connective webbing between each ampoule. In preferred embodiments, the connective webbing is used to form the ampoules into a circular shape to form an ampoule cartridge for insertion into an Ampoule Cartridge Holder (“ACH”). As used herein, a “cartridge” is two or more ampoules, which may be interconnected, or alternatively associated with, each other. Preferably, the ACH is a disk shaped enclosed container designed to hold a specific type of ampoule cartridge. In preferred embodiments, the ACH may serve one or more of the following purposes: (a) as a tamper-proof packaging container to protect cartridges during storage and transport; (b) to provide surfaces for labeling (manufacturer, type of substance, bar coding, expiry date, and prescription usage instructions); (c) to contain a gear or ratchet interface to the indexing mechanism in the device allowing the ampoule cartridge to be indexed; (d) to contain at least one component of a security key which limits the installation and operation of a cartridge holder to authorized devices and insures that cartridges are correctly loaded into the device; and (e) to contain the ampoule numbering, color coding, or Braille system for assisting the user in compliance tracking, as well as a preferable mounting location for a Radio Frequency Identification Device (RFID).

In preferred embodiments, ACHs are constructed of polystyrene, or alternative types of FDA approved medical grade plastics such as polypropylene, ABS, Nylon 6, and polycarbonate. ACHs may be of any size to accommodate the ampoule cartridge, for example an ampoule cartridge ring of an appropriate number ampoules, and may be configured, in certain preferred embodiments, to contain from 16 to 30 single-unit dose or two-unit dose ampoules. In alternative embodiments, a ACH can be of different shapes such as rectangular, square, cylindrical, circular, or pyramidal, to accommodate alternate device designs.

In preferred embodiments, a supply of ampoules, such as a ampoule cartridge ring, loaded into an ACH contains one single type of substance in each ampoule of the cartridge. Alternatively, the ampoules in the ACH may contain two or more different substances in a pre-defined order, for example to administer two or more active-ingredient-containing substances, or alternatively two or more dosage levels of the same substance.

In the preferred embodiments, the ampoule cartridge or the ACH is labeled to identify the substance, and may also include a bar code and expiry date information in a manner that is easy for the user to read while the ampoule cartridge or ACH is installed in the device and without the need to open the device. The ampoule cartridge or ACH may also use a color-coding scheme to provide a visual color reference to the user to enable them to determine the type of drug loaded into the device. Preferably, the ampoule cartridge or ACH is visible to the user while installed in the device through a large transparent window in the device. Each ampoule in the ampoule cartridge or ACH is preferably numbered on the top of the ampoule cartridge or ACH in a manner that is easily readable by the user without opening the device via a magnification window.

Eye Guide and Eye Cup

Preferably, the devices disclosed herein are positioned directly over or in the therapeutic site, for example the eye, nose, or ear, and the substance is dispensed from the center axis of the device. If the devices are used for an ophthalmic application, the substance is preferably directed accurately onto the eye of the user. In some embodiments, an attachment such as an eye cup may be used with the device to guide the dispersed substance into the user's eye and to assist in holding the eyelid in the optimum position during administration. Often times surgery leaves an area very tender, and placing an object against the area may cause the patient discomfort, or even damage the surgical area. For example, it may be preferable not to touch the eyelid or any part of the eye after eye surgery. The eye cup can help overcome this problem by guiding the dispersed substance into the eye without requiring direct contact with the eye. In certain embodiments, an eye cup is attached to a device that is designed to hold the eyelid open. The device may also designed to position the ampoules containing the substance in proper orientation with respect to the targeted site, for example the eye, ear, or nose of the user.

In preferred embodiments, a collapsible eye guide with an attached eye cup is incorporated into the devices disclosed herein. Preferably, the collapsible eye guide is extendable out of the body of the device and is locked into the ready position by a user to prepare to use the device. The eye guide and eye cup serve to position the device in the correct orientation and distance from the eye. It also facilitates opening the eyelid and holding the eyelid in the correct position during administration. In certain preferred embodiments of eye cups, there is a visual reference in the eye cup that provide a “target” for the eye to focus on during administration, for example an opening or another similar visual cue. Preferably, by focusing the eye to be treated on the opening or other visual target, the natural lens of the eye rotates upward and out of the firing path of the stream of the substance released from the ampoule, thereby facilitating correct administration of the substance to the eye.

In preferred embodiments, the eye cup incorporated into the presently disclosed devices includes an eye guide support frame configured as an open spoked frame. Preferably, one or more arms support a soft rubber or plastic eye cup that goes around the eye socket. In certain embodiments, the eye guide support arms attach to the device housing and slide to retract the eye cup into the device housing when not in use. Preferably, a user-replaceable soft plastic or rubber eye cup cover attaches to the front of the eye guide to cushion against the eyes socket and facilitate opening and holding the eyelid open during administration of a substance from an ampoule. In another preferred embodiment, the eye guide is configured as either a telescoping arm that extends by pulling out of the device and then rotating it 180 degrees to lock it into the open position, or the eye guide includes an open spoke frame that can be pulled into a fully extended position and locked into place. The telescoping eye guide supports a ring (or clip) on which is mounted the eye cup. The telescoping arm attaches to the device housing and slides to retract the eye cup when not in use. In certain embodiments, a user-replaceable soft eye cup cover attaches to the eye guide and is designed to facilitate holding the eyelid open during drug administration. The eye guide also ensures proper alignment of the eye to the device during the administration procedure.

The eye cup, and eye guide frame preferably are made from FDA approved materials. The eye cup, for example is made of soft, pliable plastic (or rubber) constructed of silicone or other FDA approved material that can be replaced by the user. In preferred embodiments, the eye cup material is transparent to enable a caregiver who may be aiding in the administration procedure to visually confirm the eyelid is open at the time the substance is dispensed. The eye guide, when fully extended and locked into the ready to fire position, preferably results in a distance between the user's eye and the release of the substance from the ampoule, for example from an ampoule nozzle, of from about 20 mm to about 30 mm. In other embodiments, locking the eye guide into the extended position also releases an integrated safety pin or alternatively a clip inside the device which enables the device to fire. Retracting the eye guide into the stowed position re-engages the safety pin which prevents the device from firing.

In preferred embodiments, a cover or cap either removable or on a pivot or hinge covers the eye guide, eye cup, and substance dispensing aperture in the body of the device to prevent contamination of the device, the substance pathway, ampoules, or accumulation of foreign matter in the device or eye cup. In certain embodiments, selected components of the eye guide, substance delivery path, and eye cup may use an antimicrobial coatings such as MICROBAN® to reduce the possibility of contamination.

The eye cup or other adapted administration device such as a nozzle for intranasal or intra-otic use may also be an integrated with the ampoule in certain embodiments of the device. For example, devices that will be used by a caregiver in an institutional care environment such as a hospital or nursing home across a wide range of users preferably incorporate individually loaded ampoules with the desired substance and a sterile eyecup or nozzle for each administration. Disclosed herein is an eyecup or alternatively a nozzle for nasal or otic delivery, mounted to a rigid plastic collar containing an ampoule and piercer for use with an embodiment of the device designed for use by caregivers to administer substances to users preferably in institutional settings. This embodiment of the ampoule integrated with an eyecup is referred to herein as an Integrated Ampoule Ophthalmic Dispenser (IAOD) or alternatively the embodiment of the ampoule integrated with a nozzle is referred to herein as an Integrated Ampoule Nozzle Dispenser (IAND). The IAOD and a self-feeding supply device that packages and loads these IAODs into the device may be used, particularly in institutional settings. When a nozzle is used, it is appropriately sized for insertion into the ear or nose. In embodiments intended for personal use, these interfaces can be reusable as there is limited concern for contamination. In embodiments intended for institutional application, however, the interface is preferably integrated with the ampoule and designed for a single use and then discarded, thereby minimizing the risk of cross-contamination between users.

UniDoserυ Drug Delivery System (UDDS)

The devices of the present disclosure, termed UniDoser™ Drug Delivery System (UDDS) by the inventors, provide a platform technology that can be adapted for a variety of therapeutic delivery applications for ophthalmic, intranasal and otic products. UDDS device configurations are determined by the following factors: (a) self-adminstration versus caregiver administration; (b) multi-dose (ampoule) refillable device versus single-dose (ampoule) disposable device; (c) single-dose versus two-dose ampoules; (d) single drug ampoule/device versus combination drug ampoule/device; (e) the mechanism of action (“MOA”) for dispensing the substance out of the ampoule by the device (mechanical versus electrically powered); and (f) the ampoule piercing mechanism (internally pierced, cutaway tip, externally pierced, burst or self-piercing). The present disclosure generally categorizes the devices disclosed herein as consumer devices or institutional devices. It is important to note that any embodiment of a device disclosed herein may be readily adapted to administer a substance from any type of ampoule disclosed herein by one of skill in the art without any undue experimentation. Thus, the device and ampoule combination chosen for administering a particular substance to a specific type of user using one of the three disclosed routes (i.e., ophthalmic, intranasal, intra-optic), will depend on factors such as the style of administration (e.g., self versus institutional administration), manufacturing costs, the route of administration, characteristics of the sample to be administered (e.g., the specific drug and drug viscosity), the desired level of sterility, and the requirements for spray plume geometry.

Certain devices of the present disclosure dispense the substance in the ampoule under pressure, for example by piston action of the plunger in the device, by simple displacement of the substance, or by expansion of air or gas (e.g., nitrogen or noble gases) in the compartment containing the substance. Preferably, the compressive force of the firing mechanism of the device is sufficient to cause the piercer to pierce the pierceable surface, as well as to disperse at least a portion of the substance from the compartment. In other embodiments, the internal mechanism of the device used to pressurize the ampoule utilizes a piston, piston-cylinder, hammer, roller or cantilever mechanism, with sufficient impact to disperse the substance in the ampoule a predetermined minimum distance. Various pressurizing directions can be applied to the ampoule, for example perpendicular or parallel relative to the release of the substance, as well as angles in between parallel and perpendicular. In certain embodiments, the compartment of the ampoule containing the substance has a space of air or gas, preferably between the liquid and the pierceable section (e.g., a head space of air or gas), which may be compressed, thereby facilitating movement of the piercer and/or the pierceable section. In some embodiments, the compression of the air or gas also facilitates release of the substance. In embodiments manufactured without a space of air or gas in the compartment, the piercer is preferably positioned in the ampoule so as to limit the degree of movement necessary to achieve piercing of the pierceable section of the compartment. In other embodiments, the piercer may have a sealing lip to provide an interference or sealing fit with the inner wall of the compartment to minimize or prevent substance from flowing between the piercer and the inner wall of the compartment.

The devices disclosed herein may be used to improve therapeutic treatments for users, whether prophylactic, post-operative, or chronic, by dispensing an active-ingredient-containing substance in a convenient and accurate manner. In preferred embodiments, the device is used after an eye surgical procedure, such as to remove a cataract or eye tumor, or alternatively after a LASIK procedure, to administer ophthalmic drugs to the eye. For example, one embodiment of the invention is used to improve post-surgical treatment of cataracts. Normally the lens of the human eye, which is made mostly of protein and water, is clear and allows light rays to pass through the lens easily. When a cataract develops, the lens becomes cloudy and opaque, and its ability to transmit light decreases. Cataract formation takes place over time and is usually caused by a change in the chemical composition of the lens. Although cataracts may be caused by trauma, intense heat, chemical burn, prolonged steroid use, or eye diseases such as glaucoma, diabetes, or tumors, eighty percent of total cataracts are senile cataracts and occur in people over the age of 50. Cataracts affect 42% of people in the U.S between the ages of 52 and 64, and 73% between the ages of 65 and 74. As the demographics in the U.S. continue to age, more people will suffer from this debilitating disease.

A cataract operation involves removing the thick cloudy lens and implanting an artificial lens in the same part of the eye. The replacement lens is much thinner than the original, and is usually made of soft transparent material that can remain in the eye for the patient's lifetime. Each year approximately 1.4 million people in the U.S. have cataract surgery and receive intraocular lens implants. Cataract surgery requires the patient to use prescribed drugs such as anti-inflammatory and anti-infectives after the surgery. The purpose of the anti-inflammatory is to reduce inflammation that begins immediately after surgery when the tissue is injured, and the purpose of the anti-infective is to prevent infection. The use of anti-inflammatory and anti-infective drugs for post-operative care after cataract surgery is the prescribed standard care, and the proper use of these drugs is of utmost importance to the patient. Standard protocols require daily administration of an anti-inflammatory or anti-infective for two to six weeks, and in the initial days following the procedure, these drugs need to be administered from two to three times each day. Unfortunately, most patients never complete the proper protocol for a variety of reasons, such as inconvenience, forgetfulness, confusion, etc. The administration of medication can also be a significant burden to many elderly people, either because they constantly question the last time they took their medicine, how much was taken, or require assistance from another person.

One of the advantages of the devices disclosed herein is that they can administer more than one drug-containing substance or more than one drug to a user either simultaneously or sequentially. This advantage is particularly useful for the post-operative treatment of cataract surgery, since the standard protocol requires that more than one category of drug is administered to the patient. With respect to the device, two drugs may be combined together in a compartment of an ampoule immediately prior to administration, and subsequently administered to the patient, or alternatively the two drugs may be in separate compartments in the same ampoule until the moment of administration, at which time they are sequentially or jointly released from the same ampoule to the patient. For example, a first compartment in an ampoule contains a lyophilized drug or a drug in dry powder form, and a second compartment contains a pharmaceutically acceptable carrier such as water or saline. The ampoule can be designed such that an area connecting the two compartments (e.g., a pierceable section) is breached (e.g., by a piercer), and the materials in the two compartments combine to, for example, reconstitute or suspend the pharmaceutically active drug in an aqueous formulation. The reconstitution or suspension of the two materials can be facilitated by, for example, a vibrator or high frequency signal generation device that vibrates the aqueous solution. After the liquid aqueous solution is formed, the drug is released from the ampoule, for example by the mechanisms disclosed herein, and administered to the user. In another preferred embodiment, the two or more drugs are present in two or more ampoules directly adjacent to each other in the ACH, and the user administrates the first drug and then either immediately or after a therapeutically appropriate length of time administers the second drug followed by any additional drugs required in the sequence.

These embodiments can be used to administer as many different substances as are needed to the patient, as well as allowing a medical profession to tailor the therapeutic administration of selected substances to maximize their therapeutic benefits for the user. For example, an ampoule cartridge or ACH may be designed that specifically provides the full regimen (prescription) of all ophthalmic drugs needed by a patient for the first several days to week after eye surgery, followed by an ampoule cartridge or ACH that complete the standard post-operative protocol. This includes prescription regimens that require a combination of different drugs that must be administered in a certain sequence at set times. Each ampoule or ACH may be identified with a color coded or numerical markings, so that the patient or person administering the drug knows when the last dosage was given, and that the next drug dose is in place for administration. The ampoule or ACH may be marked according to the time of day, as well as the drug present in the ampoule. Each day may also be marked on the ampoule itself or the ACH, so that patients know how long they have been taking the drug and when the ampoule cartridge will be empty. A colored system may also be used in which a different color indicates a different stage of treatment. For example, the device may incorporate a red light that can be seen prior to administration, which will turn green after the predetermined dose has been dispersed by the device.

Alternatively, a programmable microprocessor computer device such as an Printed Circuit Board (PCB) or an Application Specific Integrated Circuit (ASIC) combined with a Liquid Crystal Display (LCD) panel or Light Emitting Diode (LED) can be incorporate into the device to remind the patient of the appropriate time to administer the next dose of drug, how many doses remain, and to alert the user when the device should be replaced. The display can be programmed not only to show the schedule for dosage delivery, but also to have an alarm reminder go off when it is time to administer the loaded ampoule. This feature will greatly reduce the confusion many patients have over the complicated protocols involved with the post-operative or chronic care, and will result in better patient compliance and therapeutic treatment. The device can also be designed to incorporate a number of safety features, for example a delay mechanism may be included in the device to prevent the inadvertent delivery of an extra dose to the user. Another safety feature for the personal use device is a child safety lock to prevent accidental discharge of the device by a child. This device will also allow more patients to manage their own post-operative or chronic care, rather than requiring a caregiver to do so.

With respect to the post-operative treatment of eye surgery, for example cataract surgery, the device can be used to appropriately administer anti-inflammatories, anti-infectives, or steroids to the patient, in any order and sequence of time. For example, the device can administer ampoules that contain anti-inflammatory drugs and anti-infective drugs next to each other in a series of contiguous ampoules which are sequenced for back-to-back administration while in the device. After the anti-inflammatory is administered to the patient, the ACH is rotated, either manually or automatically, so that the anti-infective ampoule is administered next. The ACH continues to be rotated manually or automatically, and the ampoule dosages administered, until the ampoule cartridge is empty. Examples of ophthalmic anti-inflammatories and anti-infectives for use in post-cataract surgery care include but are not limited to tobramycin and desamethasone ophthalmic solutions, sulfacetamide sodium-prednisolone acetate, Neomycin sulfate-Dexamethasone sodium phosphate, as well as a new group of very strong anti-infective compounds called fluoroquinolones, moxifloxacin and gatifloxin.

The present disclosure thus provides a unique system for delivering preferably preservative-free unit dose systems to the front of the eye, effective for post-operative surgery using these compounds in tandem. Any of these drugs may be administered using the devices disclosed herein in a comprehensive therapeutic treatment protocol for a user. Steroids may also be administered in the same treatment regimen, merely by adding ampoules that contain the steroid to the treatment ampoule cartridge. For example, with the disclosed device a patient could administer a steroid and a fluoroquinolone as two separate drugs in contiguous ampoules, which allows the drugs to be given almost simultaneously and also mimics a combination drug. In another embodiment, an anesthetic or diagnostic aid such as fluoroscein may be enclosed in alternate ampoules with another ophthalmic drug.

Consumer Devices

Certain devices of the present disclosure are preferably designed for repeated use by a single user and for self-administration. These devices are referred to herein as consumer devices. Preferably, the device design ergonomics of a consumer device allow the device to be operable with either hand or both hands by a user. In addition, the operating components of the device are of sufficient size that they can be manipulated by an elderly or physically handicapped person with limited dexterity in their hands. This is accomplished by oversize controls and by operating actions that emphasize the use of gross motor skills over fine motor skills for most of the operating actions. In certain preferred embodiments, surface treatment of the device ensures the device does not easily slip out of the hand during operation. A consumer device preferably has a service cycle of 1200 to 1600 substance administrations before replacement. In preferred embodiments, the device components consist of FDA approved materials. Preferably, the device is constructed of high impact polystyrene or other FDA approved medical grade plastics or resins, and is “dishwasher safe,” i.e., it can be cleaned and sanitized by putting it through a conventional dishwasher cleaning cycle.

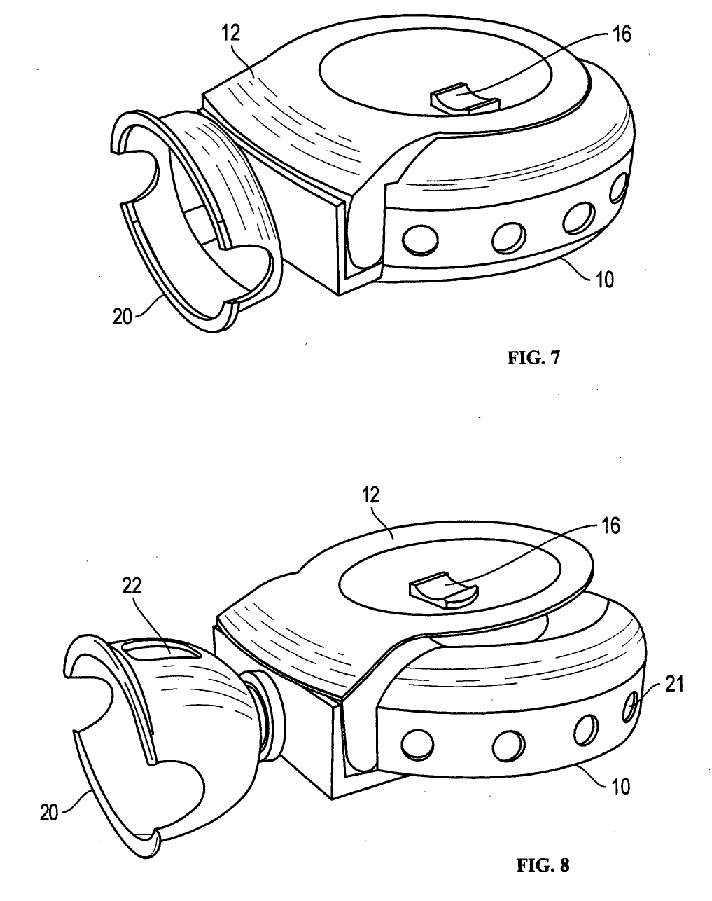

A preferred embodiment of a consumer device of the present disclosure is shown in FIG. 7. This device, termed PufferFish by the inventors, is designed to be an ophthalmic drug delivery system targeted for use by consumers for daily self-administration of a substance, for example a prescription or over-the-counter ophthalmic product, to treat conditions such as glaucoma, infection, inflammatory conditions, dry eye, or allergies, and may also be used to administer post-surgical combination drugs. The device shown in FIG. 7 is designed to fire single-dose, externally pierced ampoules, although it may be readily adapted to use with any of the other types of ampoules disclosed herein. The device opens in a clamshell fashion allowing a user to drop in an ampoule cartridge. This allows the consumer to use the device and simply reload it with a new ampoule cartridge as needed.

In preferred embodiments, an ACH for use in the device shown in FIG. 7 is an interconnected ring of ampoules, and is labeled to identify the drug, manufacturer, drug expiry date, and incorporate a bar code or Radio Frequency Identification Device (RFID) tag in a manner that is easy for the user to read while the ampoule cartridge is installed in the device without the need to open the device. The ACH preferably utilizes a color-coding or Braille scheme to enable the user to determine the type of drug loaded into the device. The ACH is visible to the user while installed in the device through a large transparent magnification window in the top, bottom, or side of the device. Each ampoule in the ACH is preferably numbered in a manner that is easily readable by the user without opening the device. Each ampoule in the ACH may contain the same drug, or alternatively each ampoule may contain one of two or more selected drugs. In addition, two-dose ampoules may also be incorporated into the current design.

In the device shown in FIG. 7, the actuator 12 is a large paddle shaped surface located on top of the device 10. The actuator is hinged 14 from the front of the housing. The actuator paddle is preferably of large size to accommodate use by the elderly. The actuator is designed to be powered by the human hand and provides a mechanical advantage to reduce the force required to fire the device (e.g., approximately 2 to 5 pounds of force to operate the paddle). The actuator paddle requires a minimum force threshold to operate to ensure that the mechanical lever delivers sufficient force to compress and fire the ampoule. During use, the actuator is released and hinges up into the ready to fire position as shown in FIG. 8. A user then presses the actuator down into the housing to fire the ampoule. A slide latch 16 in the top surface either engages the inner lip of the clamshell to hold the actuator closed when not in use or engages the dispensing mechanism. The actuator returns to the “ready” position automatically after being depressed by the user. A clasp 18 holds the clamshell in the closed and locked position at all times except when the device has to be refilled. The device in FIG. 7 is shown in the down and locked position. Ampoule Cartridge Holder and the device body are preferably molded with integral hinges and snapped together.

The device is fully enclosed to protect the interior of the device from contamination or accumulation of foreign matter. A collapsible eyecup 20 is extendable out of the body of the device and is locked into the ready position by a user to prepare to use the device. The eyecup 20 is shown in the extended position in FIG. 8. The eyecup serves to position the device in the correct orientation and distance from the eye. It also facilitates opening the eyelid and holding the eyelid in the correct position during administration. There may be a visual reference in the eyecup that provide a “target” for the eye to focus on during administration such as the opening 22. By focusing the eye to be treated on the opening or other target, the natural lens of the eye rotates upward and out of the firing path of the drug stream. Preferably, the drug-dispensing path consists of a small opening in the device body in the ready-to-fire position. In certain embodiments, a cap or cover (not shown in the Figures) can be closed over the collapsible eyecup and the drug-dispensing path to protect the device from contamination during storage and transport.

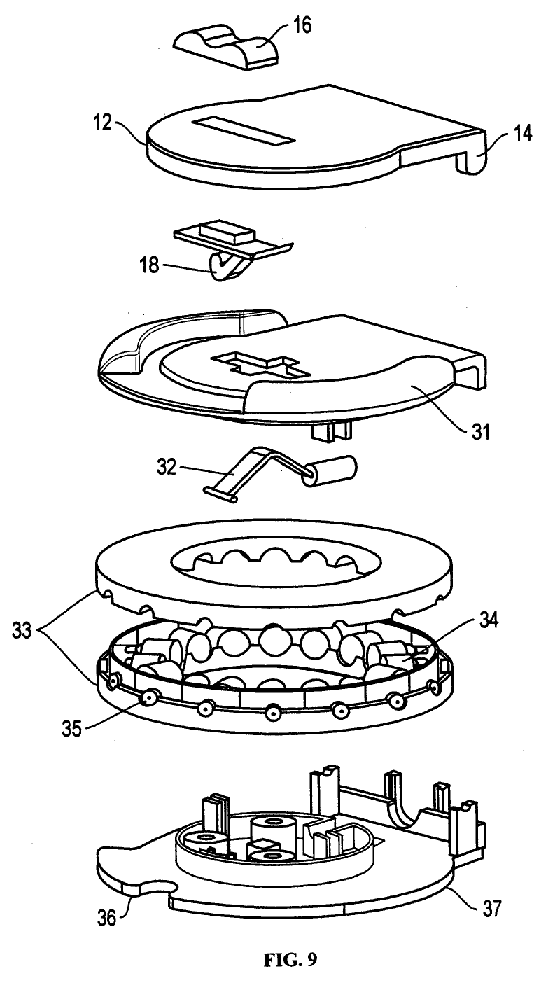

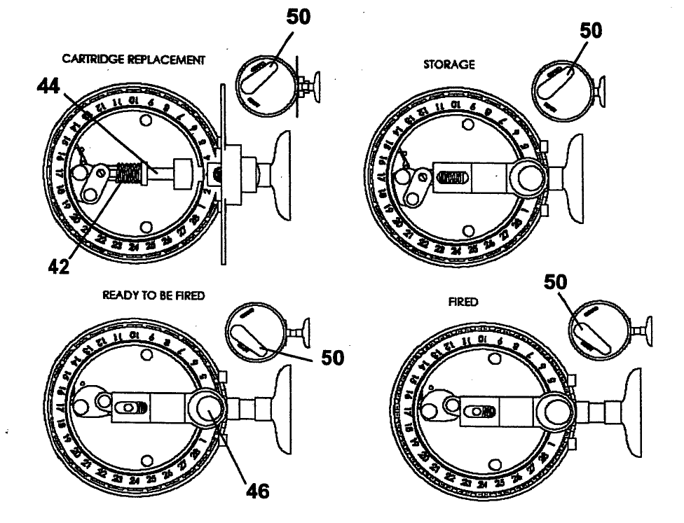

An exploded view of the device of FIG. 7 is shown in FIG. 9. The device includes a slide latch 16 that extends above the paddle shaped actuator 12 and is attached to clasp 18. When engaged, the clasp holds the actuator 12 against the top 31 of the housing and prevents firing of the device. A living hinge 32 located below the actuator is configured to depress an ampoule and release the substance when the actuator is pressed by a user. Also shown in the exploded view is a cartridge holder 33 containing the ampoule cartridge 34. The piercable portion 35 of the ampoules is exposed to the exterior of the cartridge and directs the substance into the eye cup during use. In preferred embodiments, the bottom 37 of the housing includes a magnification window 36 to enable a user to visually inspect the ampoule cartridge to determine the number of ampoules that have been used, the number of ampoules remaining, the type of substance loaded into the device, and the expiry date of the substance without opening the device.

In certain preferred embodiments, the clamshell device body is approximately 75 mm in diameter and 25 mm in height (excluding the collapsible eye guide), and the empty weight is between 65 g and 100 g. In other embodiments, the clamshell device body is preferably approximately 3 inches in diameter and 1.4 inches in height (excluding the collapsible eyeguide).

In preferred embodiments, a rotating bezel 21 on the exterior body of the device actuates the indexing mechanism. When the bezel is actuated the indexing mechanism rotates a new ampoule into the “ready to fire” position. Preferably, the indexing mechanism provides at least one positive signal to the user that a new ampoule has been properly loaded into the ready to fire position. The signal may be a tactile signal via the bezel, or a visual or auditory signal to notify the user that the device is ready to fire. For example, two forms of feedback may be provided to accommodate elderly users who may not see or hear well. The indexing mechanism can be locked in position during storage and transport so that it will not inadvertently advance the ampoule cartridge without the user's knowledge. The indexing mechanism is designed such that it only advances the ampoule cartridge or ACH in the correct (one way) direction.

In certain embodiments, the security key includes a modifiable gear or ratchet that integrates with the indexing mechanism to ensure that the ampoule cartridge or ACH loads into the device in the correct position (i.e., the number one ampoule loads into the number one firing position). Additionally the indexing mechanism may be modifiable during the manufacturing process such that only specified ampoule cartridges (e.g., representing certain types of drugs or designated manufacturer's drugs) can be loaded into the device.

The firing mechanism of the device consists of a living hinge 32 powered by the mechanical lever (actuator paddle 12). Preferably, the paddle provides a mechanical advantage for the human hand. The ampoules incorporated into the device are preferably designed to require a force of between 2 and 8 pounds to compress the ampoule and discharge the substance into the eye. The device operates to fire the substance into the eye with the correct force and volume in a range of head/eye orientations selected by the user but minimally allows the user to administer the substance when the head is held in a normal position or when laying down, where the vector of the substance dispensing spray is perpendicular to the force of gravity or in a similar direction as the force of gravity (such as when the user is reclining). Thus the device is effective to deliver the substance to the intended destination whether it fired at a vector perpendicular to the force of gravity, and from a range from 45 degrees below horizontal to directly vertical. The firing mechanism incorporates a resistance detent that requires that a minimum force threshold is applied to the paddle actuator before it will release and drive the living hinge. This feature is intended to ensure that the minimum amount of force required to compress and fire the ampoule is delivered. The actuator paddle provides a mechanical advantage of 2:1, however the mechanical advantage can be expanded to as much as 4:1 by adjusting the lever.

In order to use the device shown in FIG. 6, a user unlatches the top half of the device clamshell body. The top half of the device is hinged to swing open exposing the interior of the device and the ampoule cartridge indexing platform. In preferred embodiments, an illustrative symbol may be used on the inside of the top lid of the device to display the correct direction to align the ACH for loading. The user inserts the ACH containing the ampoule cartridge or alternatively ampoule cartridge if the holder is integrated into the device, and confirms that it is properly aligned using visual references on the device and the ACH. The top half of the device is closed and secured.

In certain embodiments, the user indexes the ampoule cartridge by moving the indexing bezel located on the outside of the device body. This procedure rotates a new ampoule into the proper firing position. The indexing system provides feedback to the user when a new ampoule has been properly positioned in the “ready to fire” position. The user feedback may be in the form of tactile feedback via the indexing lever or bezel. Other forms of feedback for the user may include visual feedback wherein the user can see the ampoule is in the correct position or alternatively a color indicator showing that the device is ready to fire, or an auditory signal such as a “click” when the ampoule is properly loaded. After the user is alerted that the ampoule is properly loaded, the user removes the eye guide cover (or cap) and extends the eye guide, which snaps into position when pulled out of the device body. Extension of the eye guide releases a safety which enables the device to be fired. The user unlocks the actuator paddle by unlatching a switch, which allows the actuator to spring open into the ready to fire position.

To fire the device, a user places the eye cup against the inner rim of the eye socket and lightly pushes to expand the eyecup to open the eyelid and hold it in the open position. The user focuses the eye on the visual target (or opening) located at the top of the eyecup to rotate the natural lens of the eye out of the path of the substance. The user then presses the actuator paddle and administers the substance. For storage, the user locks the actuator paddle into the stowed position by holding it in the depressed position and latching the switch. The user retracts the eye guide into the device body and replaces the cover or cap.

A further preferred embodiment the disclosed devices is shown in FIGS. 10. This device, termed the BlowFish device by the inventors, is also designed to be an ophthalmic drug delivery system targeted for use by consumers for daily self-administration. This embodiment may incorporate one or more of the features of the PufferFish device disclosed above. A user loads the device by opening the device in a clamshell fashion and dropping in an ampoule cartridge, which preferably contains 16 to 30 single dose ampoules. The device can be configured to fire single-dose or two-dose ampoules. The following description is for use of the device with single-dose, internally pierced ampoules, however any ampoules disclosed herein can be used with the device. Preferably, nozzles, which may be an integrated part of the device or the ampoule, face outward and are perpendicular to the circumference.

A series of cross-sectional views of the device are shown in FIG. 10 to illustrate the mechanism of the device. The four panels of FIG. 10 illustrate the device during ampoule cartridge replacement, in the ready to fire condition, in the fired condition, and in the storage configuration. A mechanically advantaged lever 50 can be seen in the Figure on the bottom of the device. This lever is connected to a spring mechanism and to the indexing system in the device. The lever has a pivot on one end and when moved by hand cocks the spring 42 into the ready to fire position. The lever is returned to the start position in the swing arc and indexes a new ampoule into the ready to fire position. Alternatively this procedure can be reversed with indexing occurring on the first movement and cocking occurring on the second movement of the lever. As shown in FIG. 10, when the ampoule cartridge is replaced, the lever is in the closed position to release the spring. The ampoule cartridge is aligned by placing ampoule #1 in the first position. After loading the ACH, the device is made ready to fire by moving the lever 50 to the ready position. The eye piece is extended and serves as a safety while the device is being cocked. When the user is ready, he or she presses the trigger button 46 to fire the device and the spring 42 pushes the piston 44 into the ampoule. After firing, the lever is moved back to the closed position and the eyepiece is retracted and covered.

As described for the previous embodiment, the clamshell device body is fully enclosed to protect the interior of the device from contamination or accumulation of foreign matter. There is a collapsible eyecup that can be extended out of the body of the device and locked into the ready position. The drug-dispensing path consists of a small opening in the device body. A cap or cover can be closed over the collapsible eyecup and the drug-dispensing path to protect the device from contamination during storage and transport. A clasp holds the clamshell body of the device in the closed and locked position at all times except when the device opened for refilling.

In preferred embodiments, the firing mechanism of the device consists of a spring that can be charged (cocked) by a mechanically advantaged, pivoting lever by the human hand. In certain embodiments, the BlowFish device is designed to fire cutaway tip ampoules. As such the firing mechanism incorporates a shielded plastic cutter blade in the front of the device which is design to cut and hold open the tip of the ampoule during the firing sequence. A trigger button activates the cutter blade and then releases a spring which drives a plunger into the ampoule to compress it and fire the substance. The cutter blade retracts and the hinged tip of the ampoule returns to its original position to plug the ampoule and reduce the potential for leakage. The cutter blade is preferably encased in a shroud which prevents exposure of the cutter to the user during the firing sequence or while loading or unloading the device. The cutter is preferably constructed of a high impact styrene plastic.

The firing sequence (from the point of triggering the device to completed delivery of the drug into the eye) is preferably complete in 200 to 300 milliseconds. In other preferred embodiments, the firing mechanism is damped to reduce any loud or startling sounds that may cause the user to blink during administrations, and to reduce vibrations within the device caused by the impact of the plunger on the ampoule. Preferably, the force required to depress the trigger button ranges from 2 to 8 pounds depending upon the type of ampoule loaded in the device and the spring configuration. The force generated by the firing mechanism can be modified by inserting springs of varying force to enable the device to fire a broader range of ampoules and substances of varying viscosity.

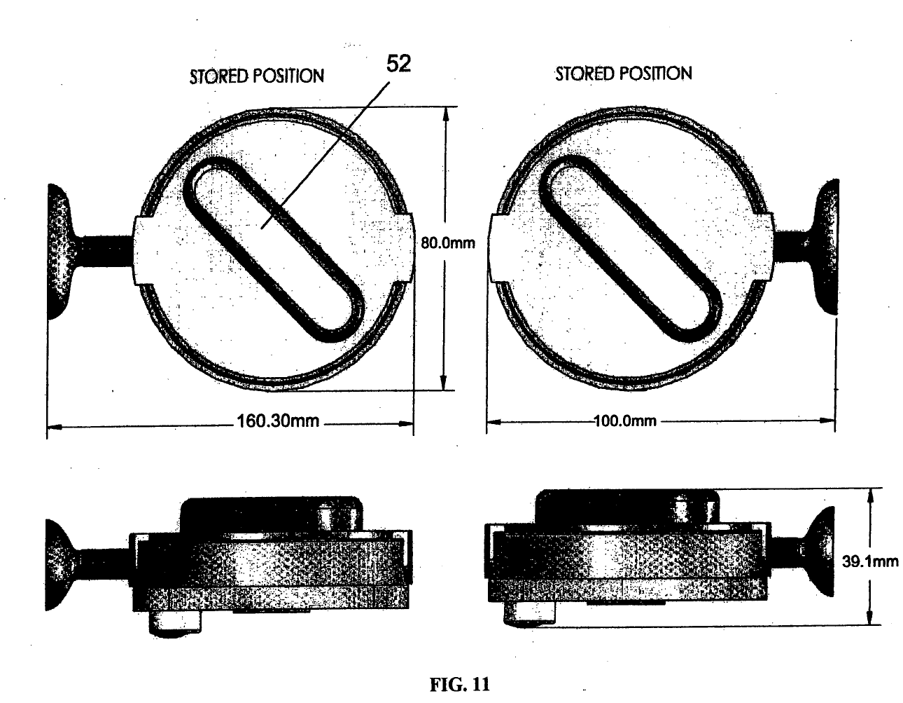

Another alternative embodiment that may include a firing system similar to that shown in the device of FIG. 10 is shown in FIG. 11. This embodiment may incorporate one or more of the features of the PufferFish and Blowfish devices disclosed above. This device uses a rotatable member 52 on the bottom of the device for cocking the mechanism. This device utilizes a mechanically advantaged inclined plane in the device to impinge on the spring mechanism and cock the device for firing. The use of the inclined plane reduces the force necessary to cock the device, making it easier for the elderly or physically handicapped to operate.

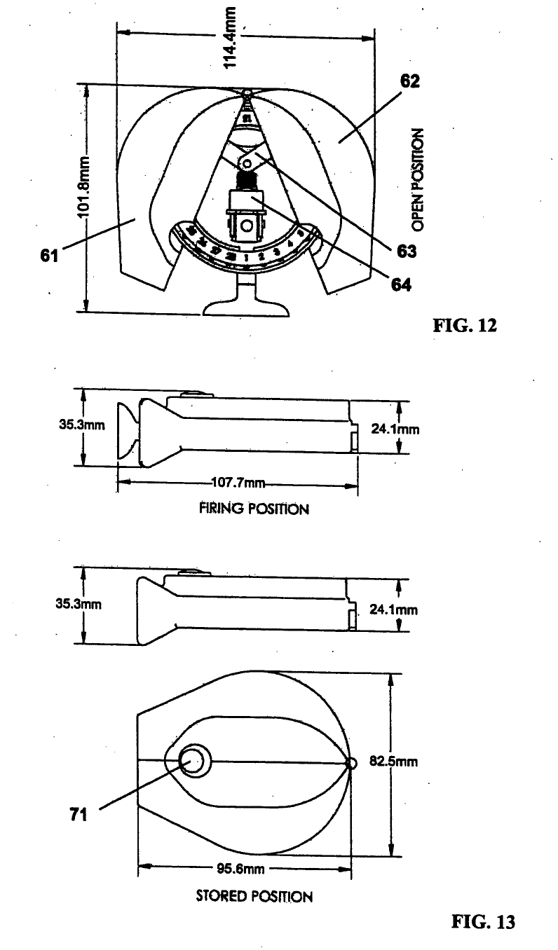

Another embodiment of a device of the present disclosure is shown in FIG. 12 and is termed the SailFish device by the inventors. This embodiment may also incorporate one or more of the features of the PufferFish and Blowfish devices disclosed above. The device includes split wings 61, 62 connected to a mechanical lever 63 in the device, which is connected to a spring driven plunger system 64. When the split wings of the device body are fully opened the mechanical lever cocks the spring. Closing the split wings indexes the ampoule cartridge and moves a new ampoule into the ready to fire position under the trigger button. Preferably, an eye cup is also integrated into the device and is extended and ready for use. The device is shown in FIG. 13 in the closed position, ready for storage. The eye cup is retracted and is covered by the wings for storage. The firing button 71 also serves as a magnified window for viewing the ampoule cartridge, which can be seen at all times by the user.

The hinged wings of the device actuates the indexing mechanism. When the wings are actuated the indexing mechanism rotates a new ampoule into the “ready to fire” position. Preferably the indexing mechanism provides several positive signals to the user that it has properly loaded a new ampoule into the ready to fire position, as disclosed above. The firing mechanism of this embodiment includes a spring that can be charged (cocked) by opening and closing the split wings of the device body. This system provides a mechanical advantage for the human hand. The SailFish device shown in FIG. 12 is designed to fire cutaway tip ampoules, but alternate designs may fire any type of ampoule disclosed herein.

In order to operate the device, a user unlatches the front of the device. The two split wings of the device are hinged to swing open exposing the interior of the device and the ampoule cartridge indexing platform. The user inserts the ACH and confirms that it is properly aligned using a visual references on the device and the ampoule cartridge. The device is closed and secured with a latch.

For administration of the substance, the user releases a latch on the front of the device to unlock the two split body wings and open the front cover over the eye cup port. The split wings are open to full extension (approximately 160 degrees) which will cock the device by pulling back the spring. The user extends the eye guide by pulling it out of the device and then rotating it 180 degrees, which locks the eyecup into the proper position. This action also releases a safety which enables the device to operate the wings to index the ACH to rotate a new ampoule into the ready to fire position. The indexing system provides feedback to the user as previously disclosed when the ampoule is properly loaded. The split wings of the body are closed and locked around the extended eyecup.

In order to fire the device, the user places the eyecup against the inner rim of the eyesocket and lightly pushes to expand the eye cup to open the eyelid and hold it in the open position. The user focuses their eye on the visual target (or opening) located at the top of the eyecup to rotate the natural lens of the eye out of the line of fire of the drug. The user then presses the firing button located on top of the device and administers the substance in the ampoule. After firing the device, the split wings of the body are opened 90 degrees and the user retracts the eye guide by rotating the eye cup 180 degrees and then pushing the telescoping arm back into the device. This action re-engages the safety and locks the eye cup into the stowed position. The split wings are fully closed, the eyecup cover is closed and the clasp is re-engaged. The device is then ready to be stowed.

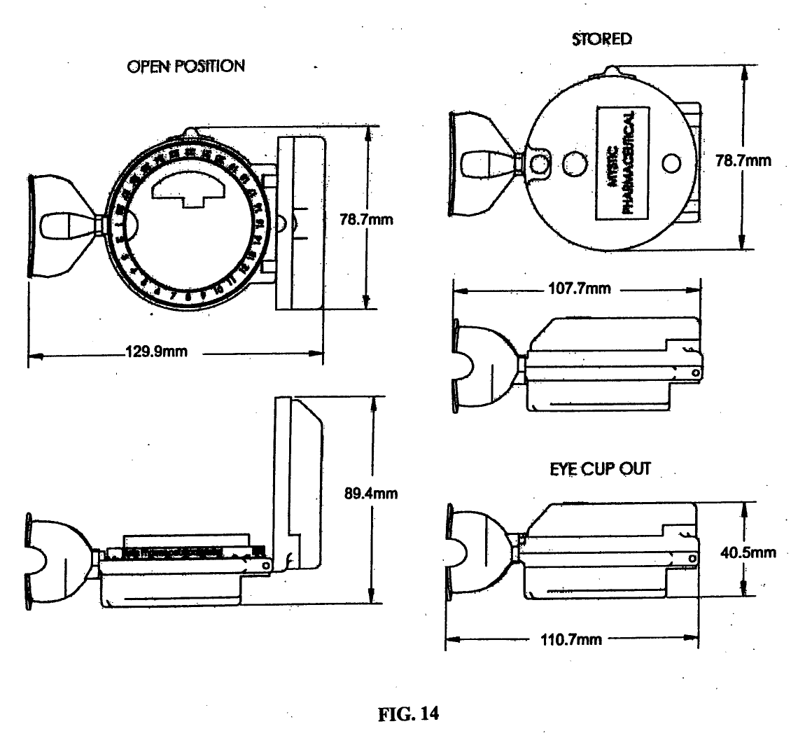

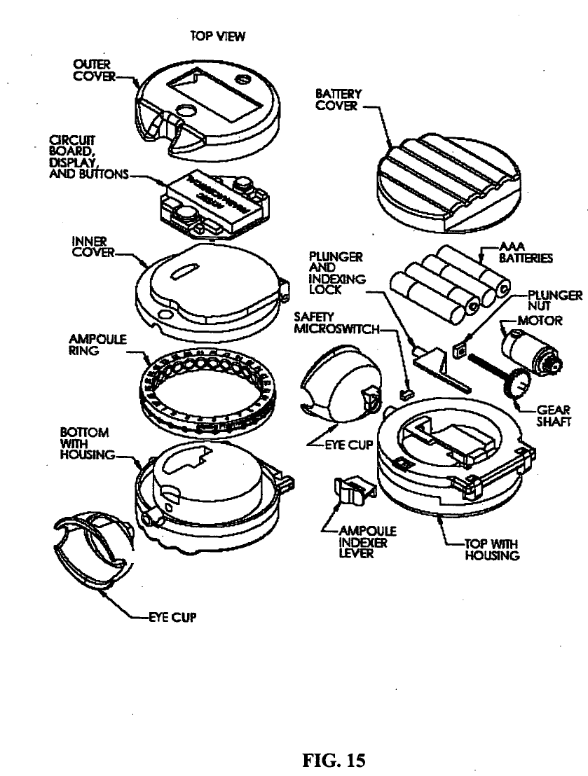

A further embodiment of the disclosed devices is shown in FIG. 14, and is termed a RockFish device by the inventors. This embodiment may also incorporate one or more of the features of the PufferFish, Blowfish, and Sailfish devices disclosed above. This device can be configured to fire single-dose or two-dose ampoules, and can also be configured to use any type of ampoule described herein. This device is battery powered, utilizing a motorized screwdrive piston as the primary mechanism of action for firing the device. An exploded view of the device is shown in FIG. 15. The device is preferably powered by conventional disposable batteries or by rechargeable batteries or any other appropriate power system. The control systems in the device for the safety mechanism, trigger, ampoule indexing release and device status reporting are electronically controlled by a programmable microprocessor computer device such as a PCB or alternatively an ASIC located in the cover of the device. A LCD panel on the exterior of the top cover provides a visual display interface for the user.

In a preferred embodiment, the device motor is a DC powered step motor or servo motor or custom motor with internal gearbox requiring 3 to 9 volts, with a torque of 40 to 120 oz-in and speed equaling 50 to 240 rpm. It is understood by those of skill in the art that this motor description is exemplary only and that other appropriate motors could be used with the device, or other devices disclosed herein.

In a preferred embodiment, the clamshell device body is approximately 3 inches in diameter and 1.6 inches in height (excluding the collapsible eyeguide). Empty weight is between 90 g and 140 g. The device body includes a magnification window to enable the user to visually inspect the ampoule cartridge to determine the number of ampoules that have been used, the number of ampoules remaining, and the type of substance(s) loaded into the device.

The firing mechanism of the device includes a step or servo style DC motor connected to a screwdrive via a gearbox. The motorized screwdrive operates at high speed to drive a plunger against the ampoule to fire the device and then retracts the plunger to a start position. Alternatively, the mechanism may include a motorized cam actuated plunger or a motorized cam/spring driven plunger. In the case of a motorized cam actuated plunger, the motor rotates a cam directly connected to the plunger to provide compressive force. In the case of a motorized cam/spring the motor actuates a cam which cocks a spring connected to the plunger. When the spring is released either mechanically or electronically, it provides the compressive force for the plunger to fire the ampoule.

In certain embodiments, any of the devices disclosed herein may incorporate a control system consisting of a PCB or alternatively an ASIC, preferably in a sealed encasement in the cover of the device. The control system performs the following functions:
On/Off device activation
LCD Display and Device Status Monitor
Electronic Trigger
Indexing Safety Release
EyeGuide Safety Release

The control system is designed to monitor and report device status including, but not limited to:
Drug Administration Sequence
Compliance Notification and Tracking
Drug Identification and Expiration Tracking
Device Operational and Lifecycle Status

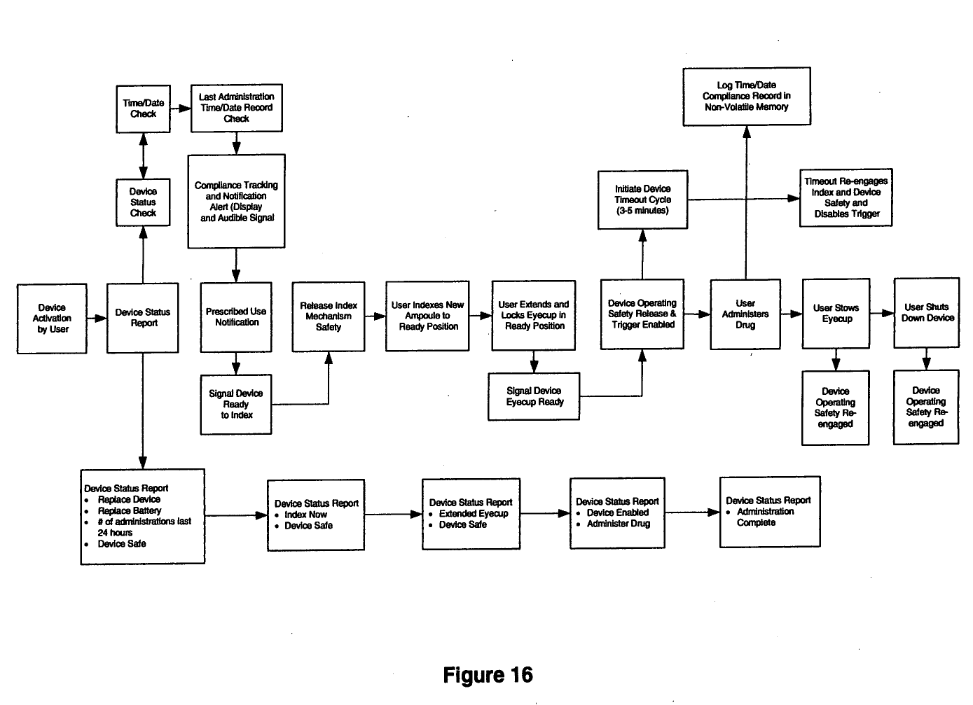

Preferably, the PCB (or alternatively the ASIC) in the device utilizes a combination of firmware, software, and non-volatile memory to operate the device, for tracking and reporting on device status and user compliance. The flowchart shown in FIG. 16 provides a summary of the primary functionality of the firmware/software technology for battery powered devices disclosed herein.

The first generation firmware/software capabilities of electrically powered devices primarily provides the following key functions:
Device Status Reporting—device lifecycle, battery status, time/date clock.
Operating Sequence Reporting—the capabilities to determine the current state of the device in terms of the operational process and next procedural steps and notify the user via display interface or audible signals.
Compliance tracking—number of administrations in past 24 hours, last administration time/date record.
Control of safety mechanisms for indexing the device and firing the device.
Automated shutdown procedure.

More advance software/firmware in the device coupled with a field programmable ASIC or similar chip mounted in the ampoule cartridge yields the following additional capabilities:
Track and record drug administration and detailed compliance history for a particular user and prescription or therapeutic treatment.
Determination whether the correct ampoule cartridge and substance is loaded into the device and that the ampoule cartridge is the correct prescription for a given user.
Track and confirm whether the substance(s) administered to the user will have any adverse reactions with any other medications currently prescribed to the user.
Determine whether the substance(s) in an ampoule cartridge have expired.
Automated notification for the user when it is time to take their drug based upon individualized prescriptions loaded into the ampoule cartridge chip by the physician, third party formulator, or pharmacist.
Prevent device operation if the user is not following prescribed use or in the case of abuse.
Upload an individual's compliance history into medical records or for billing purposes.
Automated re-order of prescription refills.

The described device is configurable to fire either single-dose or two-dose ampoules. Two-dose ampoules require modification to the PCB control system to program the motorized screwdrive to compress one half of the two-dose ampoule with the first press of the trigger and then the remainder with the second press of the trigger. The motorized drive compresses and fires the first dose, then relieves the pressure on the ampoule by backing up the plunger or roller to relieve compression of the ampoule to prevent inadvertent leakage. Administration of the second dose is completed when the trigger is pressed a second time and the ampoule is fully discharged.

In certain embodiments, for safety of operation, there is a micro switch connection mounted on the eye guide. When the eye guide is extended to place the eye cup in the “ready” position, the micro switch releases a safety mechanism which enables the device to be fired. Collapsing the eye guide/eye cup back into the device re-engages the safety mechanism. In the event the device is left in a fully enabled position (i.e., device enabled and eye cup in the extended position) for a period of more than 3 minutes (this time limit can be varied), a safety circuit will shut the device down and it will not fire unless reactivated. A clasp holds the clamshell body of the device in the closed and locked position at all times except when the device has to be refilled.

To use the device described above, a user presses a button on the top of the device to activate and enable the device. This step releases a small cam or gear that locks the ampoule cartridge in a safe position during storage and transport. The user indexes an ampoule into the “ready to fire” position by sliding a small lever on the side of the device. The user may receive feedback in several forms to confirm the device has completed an index step. For example, when the lever stops at the end of the index step, the device emits an audible mechanical or electronic signal, and visually the ampoule number on the top of the cartridge can be viewed through the magnification window on the top of the device. Alternatively, the LCD or LED panel can display an icon or text signal that the device is ready to fire but still in a “safe” mode.

Upon completion of the firing sequence the ACH safety reengages electronically by reengaging the cam or gear that locks the cartridge and prevents it from moving. The user then, for example, retracts the eye guide by rotating the eyecup 180 degrees or releasing the catch and then pushing the telescoping arm back against the device. This action re-engages the device operating safety and locks the eye cup into the stowed position. The user can deactivate the device by pushing the on/off button or the device automatically deactivates after approximately three minutes of non-use (or any other desired amount of time). The cap or eye cup cover is replaced and the device can be stored.

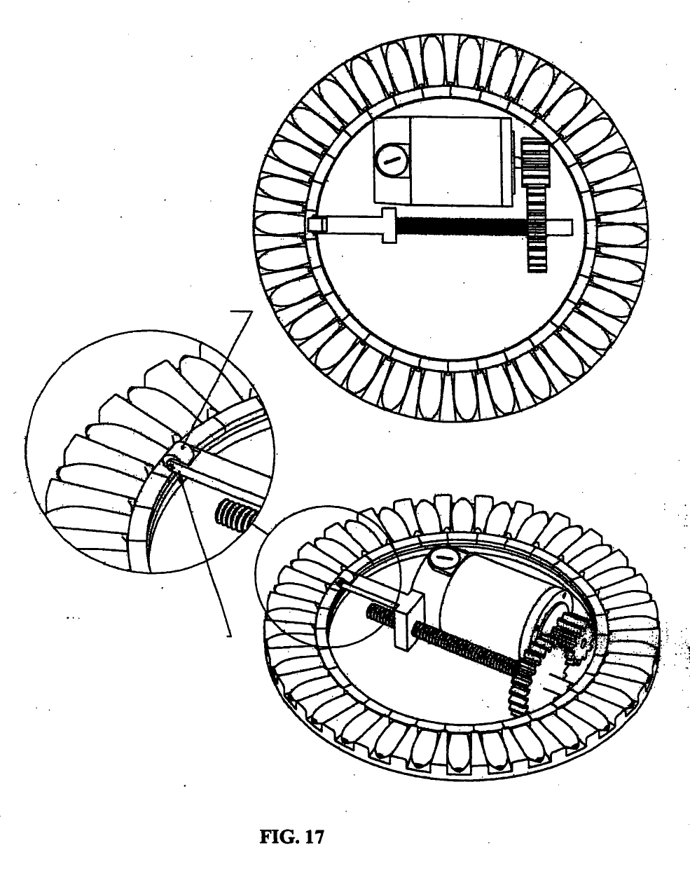

A further embodiment of the disclosed devices is shown in FIG. 17, and is termed a BoneFish device by the inventors. This embodiment may also incorporate one or more of the features of the PufferFish, Blowfish, Sailfish, and RockFish devices disclosed above, and is similar in design and construction to the RockFish device. This device can be configured to fire single-dose or two-dose ampoules, and can also be configured to use any type of ampoule described herein. Preferably, this device is designed for firing single-dose, burst ampoules, and a replaceable burst ampoule cartridge, which is designed to hold the unique shape of the burst ampoules securely, is loaded into the device. This device is battery powered, utilizing a motorized screwdrive roller as the primary mechanism of action for firing the device. The device is preferably powered by conventional disposable batteries or by rechargeable batteries or any other appropriate power system. The control systems in the device for the safety mechanism, trigger, ampoule indexing release and device status reporting are electronically controlled by a PCB (or alternatively an ASIC) chip located in the cover of the device. An LCD panel on the exterior of the top cover provides a visual display interface for the user.

The firing mechanism of the device consists of a step or servo style DC motor connected to a screwdrive via a gearbox. The motorized screwdriver operates at high speed to drive a roller over the burst ampoule to fire the device and then retract the roller to a start position. In preferred embodiments, as the roller passes over the burst ampoule, it compresses and bursts a scored opening at the tip of the ampoule and squeezes the fluid in the burst ampoule to the front and out the scored opening. In another embodiment, the roller mechanism can be replaced by a curved rocker which compresses and rocks or wedges over the top of the ampoule.

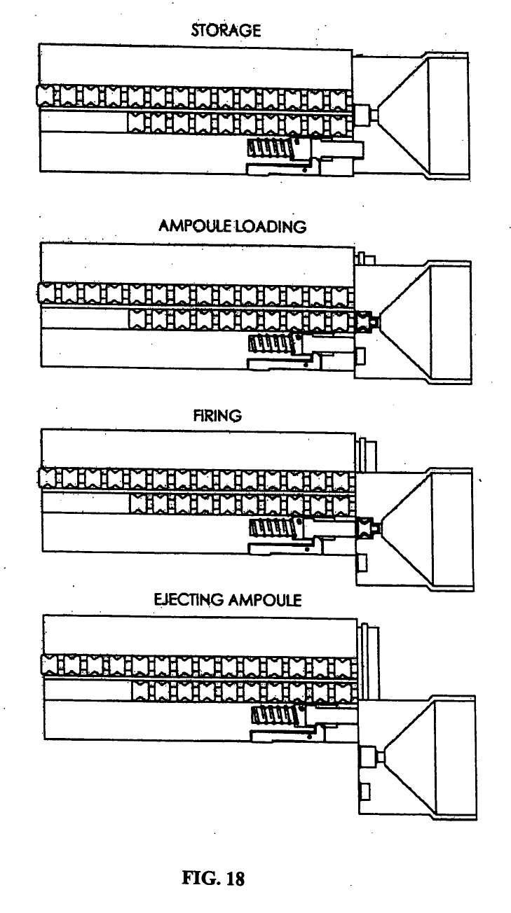

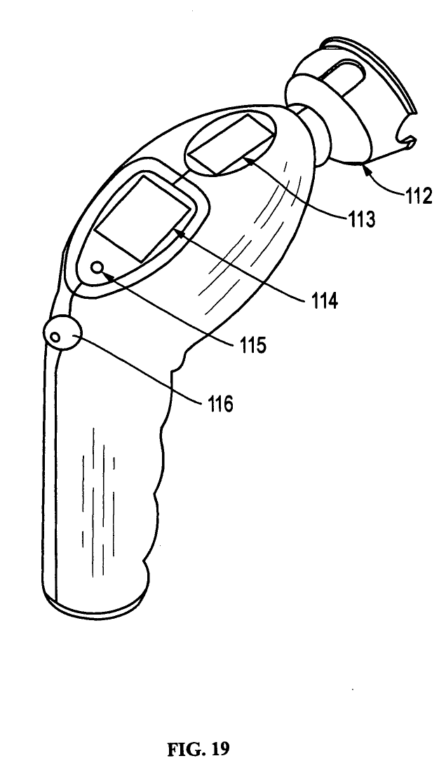

Another embodiment of a consumer device is shown in FIG. 18, and is termed a Slider device by the inventors. A series of cross-sectional views of the device in FIG. 18 illustrate the mechanism of the device. The four panels of FIG. 18 illustrate the device during storage configuration, the ready to fire condition, the firing condition, and the ejecting ampoule condition. While this embodiment may incorporate one or more of the features of the PufferFish, Blowfish, Sailfish, RockFish, and Bonefish devices disclosed above, this device is distinct in design from these other devices in that it consists of a cylindrical, oval, or rectangular shaped tube with an eye cup located at one end of the tube. The device as disclosed herein is designed for firing single-dose, internally pierced ampoules, but can be configured to fire both single-dose and two-dose ampoules, as well as any type of ampoule described herein.

In preferred embodiments, the device body incorporates a transparent window to enable the user to visually inspect the ampoule cartridge to determine the number of ampoules that have been used, the number of ampoules remaining, and the type of substance loaded into the device. The ampoule cartridge may be loaded into the device through a small access port at the base of the device to expose the one or more cartridge chambers of the device. In preferred embodiments, the user loads an ampoule cartridge into the chamber, wherein the ampoule cartridge incorporates a flange or tab on the top of the ampoule cartridge, thereby requiring the user to insert the ampoule cartridge in the proper orientation in the device (e.g., with drug labeling and ampoule numbering being displayed so it can be view through a transparent window in the device by the user to aid in compliance tracking and drug identification). After the ampoule cartridge is inserted into the chamber, the access port is closed and latched to secure the ampoule cartridge in the device. This process is repeated to load each additional chamber in the device. Preferably, the ampoule cartridge contains approximately 10 to 16 single-dose ampoules. In certain embodiments, the ampoules are aligned end to end, or alternatively are stacked horizontally in one or more adjacent rows. In preferred embodiments, two or more independent storage chambers are present in the device, which allows two or more different substances to be stored in the device. In other embodiments, the ampoule cartridges are installed in the end of the device and are graduated to show the remaining number of ampoules in each storage chamber.

The body of the device is fully enclosed to protect the interior of the device from contamination or accumulation of foreign matter. Preferably an eye cup sits on the top of the device, and a plunger locks the eye cup to prevent it from sliding in storage. The eye cup slides horizontally by pressing it with a thumb or fingers to move an ampoule into the loading, firing, and ejecting positions. The device is loaded by inserting one or more ampoule cartridges in the form of a cylindrical tube containing a supply of ampoules into the bottom of the device. Indexing an ampoule in to the ready to fire position entails sliding the eye cup over one of the two or more ampoule cartridge chambers and turning the device upside to allow an ampoule to be gravity fed into the ready to fire position in the eye guide. Alternatively, the ampoule can be fed into the eye guide using a spring or lever to move the ampoule from the ampoule cartridge into the ready to fire position. Releasing the eye cup allows it to slide back over the plunger. Alternatively, the eye cup is configured with a spring that returns is to the ready to fire position when released by the user. In preferred embodiments, a mechanical lever on the side of the device is connected to a spring driven plunger mechanism. The lever is pulled toward the base of the device which cocks the spring and plunger into ready to fire position. When released by a mechanical trigger the spring drives a small plunger to compress and fire the ampoule that has been loaded into the ready to fire position. After the substance of the ampoule is released, the ampoule is ejected by sliding the eyecguide in the opposite direction to expose the spent ampoule. The user gently shakes the device to dislodge the ampoule from the eye guide, disposes the spent ampoule, and returns the eye guide to the ready to stowed position. The cap or cover is replaced and the device is stowed.

In preferred embodiments, the device has a service cycle of 400 to 600 drug administrations before replacement. In addition, the device preferably is constructed of FDA approved plastics such as high impact polystyrene, polycarbonate, ABS, or Nylon 6. Preferably the device is approximately 25 mm in diameter and 130 mm in length. Empty weight is between 45 g and 80 g.

Institutional Devices

Certain devices of the present disclosure are preferably designed for use by caregivers administering substances to patients or residents in an institutional setting such as clinics, hospitals, nursing homes, and assisted care living environments. Caregivers in these situation must often administer over-the-counter or prescription ophthalmic products for a variety of conditions, such as glaucoma, dry eye, and allergies, and often must administer anti-infective, anti-inflammatory, and post surgical combination drugs. These devices are referred to herein as institutional devices.

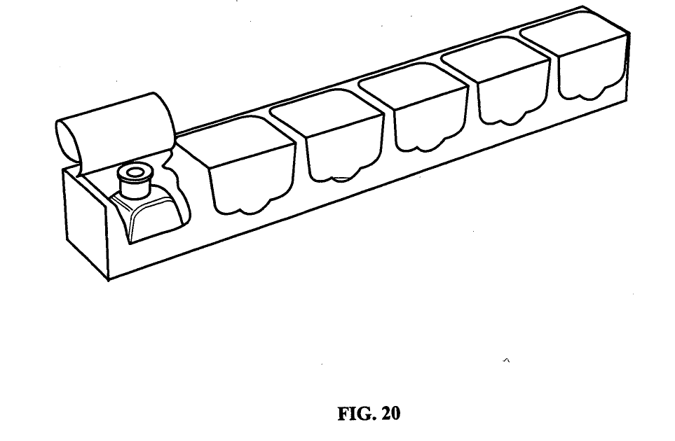

An embodiment of an institutional device is shown in FIG. 19, and is termed the SeaHorse device by the inventors. This device is designed to be an ophthalmic drug delivery system for broadly administering an ophthalmic drug to a patient population. The device includes an eye cup 112, a device status indicator display 113, device/ampoule/patient information display 114, a display button for menu scroll 115, and a multi-function thumb button 116 for menu selection, grapple open, grapple close, and safety. The device can be configured to fire single dose or two-dose ampoules. The following description is directed to a configuration for firing single-dose, externally pierced ampoules, although the device may be adapted to use any of the ampoules described herein.

The SeaHorse is preferably a battery powered device utilizing a motorized screwdrive piston as the primary mechanism of action for firing the device. The device is powered by conventional disposable batteries or alternatively rechargeable batteries or other suitable power source. The control systems in the device for the safety mechanism, trigger, ampoule loading and eject, and device status reporting are electronically controlled by a PCB or ASIC chip located in body of the device. Two LCD panels on the exterior of the top cover provide a visual display interface for the caregiver.

The device body is fully enclosed to protect the interior of the device from contamination or accumulation of foreign matter. There is a grapple mount on the front of the device designed to grasp, hold, and eject a single use Integrated Ampoule Ophthalmic Dispenser (“IAOD”). The IAOD consists of a single use eye cup, an ampoule containing a desired substance, and a mounting collar which holds the entire component together and serves as the means to mount the IAOD onto the device for administration of the substance. Preferably, a soft plastic (or rubber) eyecup is affixed to the rigid collar. Alternatively, an ampoule can be integrated with a mounting collar and a nozzle designed for use in nasal or otic drug delivery. This configuration, which is termed Integrated Ampoule Nozzle Dispenser (IAND), consists of a single use nozzle incorporating an internal, movable plunger or piston, an ampoule containing the substance, and a mounting collar which holds the entire component together and serves as a means to mount the IAND onto the device for administration. These dispensers may be used to administer a desired substance to the eye, nose, or ear of a patient.

In preferred embodiments, the IAOD facilitates opening the eyelid, holding the eyelid in the correct position during administration, and positioning the device in the correct orientation relative to the eye. For example, notched cutouts on both sides of the eyecup cause the eyecup to flex and open the eyelid when gentle pressure is applied. There is a visual reference in the eyecup that provide a “target” for the eye to focus on during administration such that the natural lens of the eye rotates upward and out of the firing path of the substance stream. In addition, the eye cup material is preferably transparent. This feature is designed to enhance the caregiver's ability to view the patient eyelid position during administration of the substance. The IAOD is designed to be used for a single administration (or two sequential administrations in the case of a two-dose ampoule) and then discarded.

In preferred embodiments, the device has a service cycle of 20,000 substance administrations before replacement. In other preferred embodiments, the device body is approximately 7 inches in height, 5 inches long (including the IAOD) and 2 inches in depth. Empty weight is between 1 and 2 pounds. Preferably, the device body and housing elements are constructed of an FDA approved material such as polystyrene, polycarbonate or ABS plastic. In preferred embodiments, the device is powered by a DC powered step motor, servo motor, or custom motor with internal gearbox requiring 3 to 9 volts and with torque of 40 to 120 oz-in and speed equaling 50 to 240 rpm. It is understood by those of skill in the art that this motor description is exemplary only and that other appropriate motors could be used with the device, or other devices disclosed herein.

In certain embodiments, the IAOD or IAND are packaged in either individualized foil sealed containers or in a spring fed cartridge containing multiple dispensers. The packaging system is an integral component of the system because it is designed to enable the caregiver to load the device without having to come in physical contact with the IAOD or IAND either for loading or ejection, which reduces the risk of cross-contamination. An example of such a design is shown in FIG. 20.

The device can be operated with either hand. The handle is contoured and has a textured surface to make it more comfortable to grip and to enhance handling of the device during operation. The handle orientation of the device ranges from a 90 degree angle relative to the barrel, to a swept back position of 120 degrees. An alternate configuration for the handle includes the capability to pivot or rotate the handle from 0 to 180 degrees to enable the device to be used more conveniently by the caregiver for intranasal drug delivery.

In preferred embodiments, the device utilizes a combination of visual displays (LCD panel or LED indicators) and audible signals to communicate device status, operational status and patient information. For example, there are two LCD display panels located in the tip of the device.

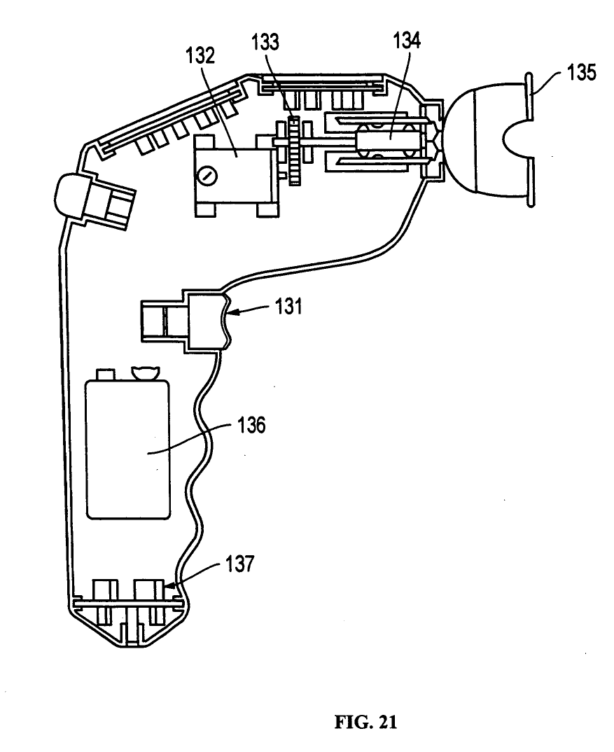

A cross section view of the device is shown in FIG. 21. The device includes a trigger button 131 that functions to fire the ampoule. The DC motor 132 is connected to a screw driver gear 133 that presses the ampoule 134 and fires the contents of the ampoule through the eye cup into the eye 135. Also shown in the figure is the battery 136 and an optional bar code reader/RFID scanner 137. The bar code scanner or RFID Scanner enables the device to identify substances, users (patients or drug recipients), and records their administration history (compliance). Additionally, the device may be enabled to communicate either wirelessly or via a docking cradle to medical records management systems and billing systems. The preferred embodiment of the device is designed to use a standard 9 volt battery which can be either disposable or rechargeable. Alternative battery configurations are also possible to accommodate international standards or higher power requirements.

An electronically controlled grapple on the tip of the device is designed to securely grasp the mounting collar of the IAOD or IAND and secure it to the tip of the device for administration. The grapple consists of two, three, four, or more plastic, plastic laminated, or metal clips or hooks, which are integrated into the barrel of the device. When the grapple is activated by the press of the control button on the back of the device by the caregiver it opens and extends slightly. The caregiver positions the tip of the device on the mounting collar of the IAOD or IAND and closes the grapple by pressing the primary control button with his or her thumb a second time. The grapple electrically cycles and locks onto the mounting collar of the IAOD or IAND drawing it into the correct firing position. A micro switch located in the tip of the device confirms the IAOD or IAND is properly loaded onto the device. If the IAOD or IAND is misaligned the device provides an error message to the caregiver and the procedure can be cycled again. When the IAOD or IAND is properly loaded the micro switch sends a signal to the PCB or ASIC. The caregiver receives a visual and audible signals confirming the “ready” status of the device. The PCB or ASIC then releases the safety and enables the electronic trigger of the device.

Following completion of the administration procedures the caregiver can eject the spent IAOD by actuating the grapple via the control button. The grapple releases and ejects the ampoule which is discarded.

The firing mechanism of the device includes a step or servo style DC motor connected to a screwdrive via a gearbox. The motorized screwdriver operates at high speed to drive a plunger against the ampoule to fire the device and then retract the plunger to a start position. Alternatively the mechanism of action can include a motorized cam actuated plunger or a motorized cam/spring driven plunger. In the case of a motorized cam actuated plunger the motor rotates a cam directly connected to the plunger to provide compressive force. In the case of a motorized cam/spring the motor actuates a cam which cocks a spring connected to the plunger. When the spring is released either mechanically or electronically, it provides the compressive force for the plunger to fire the ampoule.

As previously disclosed herein, certain embodiments of the institutional devices disclosed herein may incorporate a control system consisting of a PCB or alternatively an ASIC, and perform one or more of the functions previously outlined, utilizing a combination of firmware, software, and non volatile memory to operate the device. In addition, the control system may control the firing sequence of the device, including loading and unloading IAODs, and operating the bar code scanner or RFID scanner. A rotating mouse button located near the two LCD panels allows menu scrolling and menu selection for device status controls.

As stated above, institutional devices may be adapted to fire either single-dose or two-dose IAODs. Two-dose ampoules require modification to the PCB control system to program the motorized screwdrive to compress one half of the two-dose ampoule with the first press of the trigger and then the remainder with the second press of the trigger. The motorized drive compresses and fires the first dose, then relieves the pressure on the ampoule to prevent inadvertent leakage and then completes the process when the trigger is pressed the second time. The capability to administer two doses from a single ampoule provides a further reduction of costs to the consumer.

In other embodiments, there is a micro switch connection mounted on grapple in the device. When the device is properly loaded with an IAOD, a micro switch in the tip of the device releases a safety mechanism and enables the electronic trigger which allows the device to be fired. Aborting the firing sequence prior to administration can also be accomplished via the device status mouse/scroll button. In addition, a time out circuit in the device will deactivate the device and engage the safety mechanisms if the device is not used for a certain period of time, for example 3-5 minutes.

In preferred embodiments, the caregiver activates the device by pushing the thumb button on the back of the device which turns on the device. If the device is configured with a bar code scanner, the caregiver scans the patient's bar code using the scanner located in the handle of the device. The bar code scanner is activated by pushing and holding the thumb button. The bar code scanner is then used again to scan the bar code of the ampoule to be administered. This information is logged into the memory on the device or alternatively is transmitted wirelessly to a medical records management system. In the former case the patient compliance data is later downloaded when the device is docked in its cradle and synchronized.

To fire the device, the caregiver places the eyecup against the inner rim of the eye socket and lightly pushes to expand the eyecup to open the eyelid and hold it in the open position. The user focuses his or her eye on the visual target (or opening) located at the top of the eyecup to rotate the natural lens of the eye out of the line of fire of the substance. The care giver presses the trigger button located in the handle of the device and administers the substance. In the event the device is loaded with a two-dose ampoule this procedure is repeated a second time on the alternate eye. The IAOD is discarded by pressing the thumb button following completion of the administration.

Intranasal Devices

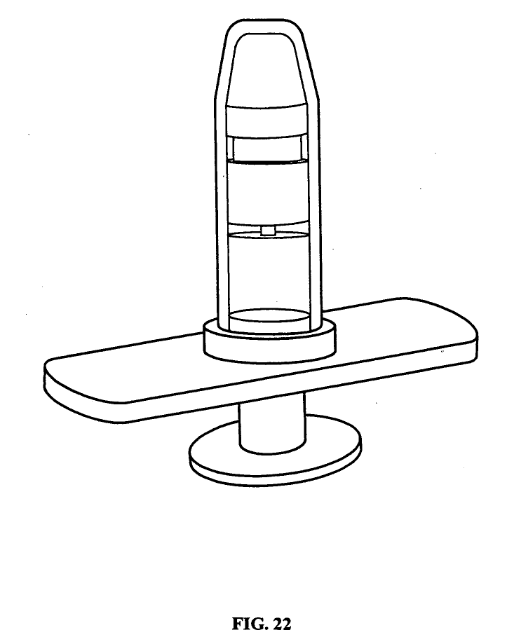

An embodiment of an intranasal device is shown in FIG. 22, which is termed the Swordfish device by the inventors, is designed for either individual use or institutional use. This device is a single use, disposable intranasal drug delivery system, preferably for use with infrequently administered prescription drugs or vaccines. Illustrative applications for the device include intranasal delivery of pain medications (such as those used for migraine headaches) or influenza vaccines. The device consists of the following primary components: a doser (the tube shaped housing in which the plunger and ampoule reside), a plunger, and an ampoule containing the substance to be administered. The device is fired by pushing the plunger into the doser. This device can be readily adapted to administer any type of ampoule disclosed herein, as well as single-dose or two-dose ampoules. Preferably the device is constructed of FDA approved materials such as polystyrene or polypropylene.

In preferred embodiments, the plunger and inner wall of the intranasal device incorporate a geometrically shaped ridge guide that allows the plunger to be locked in a fixed position for packaging and transport. To use the device, the user pulls out and rotates the plunger from the closed and locked position until the raised ridge of the plunger clicks and locks into the single-dose firing position. For example, the plunger can be rotated 180 degrees to unlock the device and enable it to fire an ampoule, for example a single-dose ampoule. The user may visually confirm that the plunger is properly positioned by, for example, aligning a mark on the plunger with a mark on the doser. Once the device is ready to fire, the user inserts the tip of the doser into the appropriate nasal cavity and fires the device by depressing the plunger until it comes to a full stop position.

The device can also be configured to fire a two-dose ampoule, for example when the prescribed therapeutic application requires administration into both sides of the nose. To administer a two-dose ampoule, the same procedure is followed as for the single-dose ampoule, and after the first dose is fired the user withdraws the device and rotates the plunger to a second two-dose firing position. For example, this is accomplished by rotating the plunger 90 degrees to fire the first half of the two-dose ampoule, and then rotating the plunger an additional 90 degrees to fire the second half of the two-dose ampoule. The user inserts the tip of the doser into the alternate nasal cavity and fires the device by depressing the plunger until it comes to a full stop position.

In certain embodiments, spray plume geometry and droplet size administered by the intranasal device are controlled by the design of the ampoule piercer. The preferred embodiment is an external piercer with an internal hollow channel ranging in size of about 0.28 mm to about 0.75 mm internal diameter. The internal diameter of the exit point or nozzle of the internal channel can be the same as the internal diameter of the hollow channel, or alternatively can be varied by making it smaller to modify the shape or droplet size of the spray plume as it exits the device. In other embodiments, the device incorporates the use of an external piercer with an internal channel and nozzle to form a channel that is not a straight path, but rather has been angled to direct the spray plume of the emitted drug to strike directly the nasal mucosa. Redirecting the spray plume toward the nasal mucosa is achieved by angling the internal channel of the external piercer between 20 to 90 degrees relative to the straight-line shaft of the doser and plunger mechanism. The tip of the dosing tube is also modified to incorporate a comparably angled opening to enable the spray plume to exit the doser at 20 to 80 degrees from the angle of insertion of the device into the intranasal cavity.

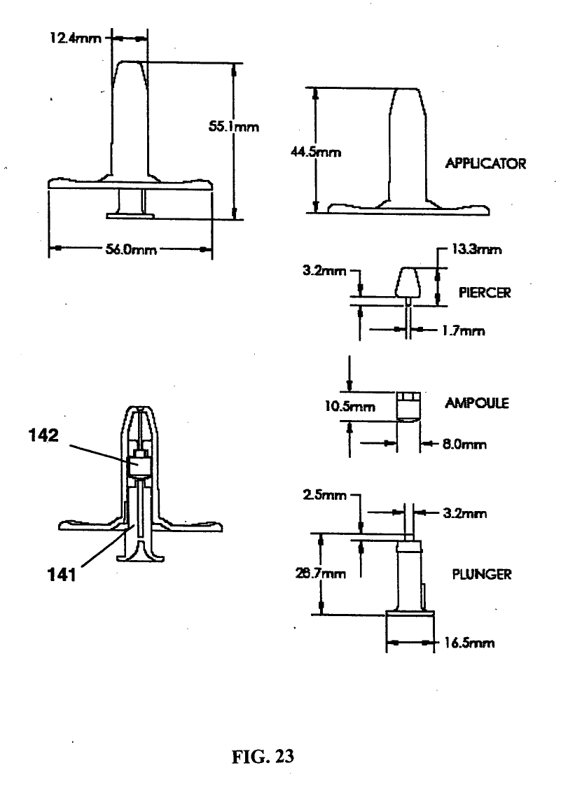

Another embodiment of an intranasal device is based on the previously described SeaHorse device, in which the device is configured to administer certain substances into the nose or ear using the IAND. The IAND is a single use ampoule applicator that attaches to the device in the same manner as the IAND (i.e., using a rigid mounting collar). The IAND includes an applicator tube, an ampoule, and a movable plunger. The IAND can be configured for example with either internally pierced ampoules or externally pierced ampoules, or any other types of ampoules disclosed herein. A cross-sectional view of a plunger 141 and internally pierced ampoule 142 is shown in FIG. 23.

The IAND mounts on the end of the device in the same manner as previously described for the IAOD. An alternative design of the ampoule for intranasal delivery enables the spray plume to be dispensed in a concentrated pattern at angles ranging from 60 degrees to 90 degrees from the direction of entry of the tip of the IAND into the nose. This capability is designed to direct the spray plume pattern to mucosa within the nasal cavity.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the methods described herein without departing from the concept, spirit and scope of the invention. More specifically, it will be apparent that certain agents that are chemically or physiologically related may be substituted for the agents described herein while the same or similar results would be achieved. All such similar substitutes and modifications apparent to those skilled in the art are deemed to be within the spirit, scope and concept of the invention as defined by the appended claims.

What is claimed is:

1. A drug delivery device comprising:
a housing configured to contain one or more ampoules comprising an active-ingredient-containing substance in a compartment, wherein the ampoules comprise a piercable region in the compartment;
a firing mechanism effective to apply pressure to an ampoule effective to cause the ampoule to be pierced at the piercable region and to release the substance under pressure through the piercable region of the compartment;
a delivery device configured to direct the released substance to the eye, nasal passage, or ear canal of a user;
a mechanically advantaged mechanism whereby a user positions the device into a ready to fire configuration; and
a mechanically advantaged firing control whereby a user activates the firing mechanism to release the substance from the ampoule.

2. The drug delivery device of claim 1, wherein the substance comprises an active pharmaceutical ingredient.

3. The drug delivery device of claim 1, wherein the substance is preservative-free.

4. The drug delivery device of claim 1, wherein the substance is atomized or aerosolized upon release from the ampoule.

5. The drug delivery device of claim 1, wherein the substance is sterile until released from the ampoule.

6. The drug delivery device of claim 1, wherein the ampoule is a single-dose ampoule or a two-dose ampoule.

7. The drug delivery device of claim 1, wherein the ampoule is selected from the group consisting of internally pierced ampoules, cutaway tip ampoules, externally pierced ampoules, burst ampoules, and self-piercing ampoules.

8. The drug delivery device of claim 1, wherein the ampoule is pierced externally.

9. The drug delivery device of claim 1, wherein the ampoule is pierced internally.

10. The drug delivery device of claim 1, wherein the ampoule further comprises a head space of gas or air.

11. The drug delivery device of claim 1, wherein pressure is applied to the ampoule by a piston, plunger, or roller.

12. The drug delivery device of claim 1, wherein the firing mechanism is a hinge, spring, cam, or motorized drive.

13. The drug delivery device of claim 1, wherein the ampoules are interconnected.

14. The drug delivery device of claim 1, wherein the ampoules are in an Ampoule Cartridge Holder.

15. The drug delivery device of claim 14, wherein the Ampoule Cartridge Holder comprises an indexing orientation key.

16. The drug delivery device of claim 1, wherein the device further comprises a programmable microprocessor.

17. The drug delivery device of claim 16, wherein the programmable microprocessor is a Printed Circuit Board or an Application Specific Integrated Circuit coupled to visual display interface and audible notification system.

18. The drug delivery device of claim 17, wherein the visual display interface is a Liquid Crystal Display or Light Emitting Diodes.

19. The drug delivery device of claim 1, wherein the force required by a user to cock the device into a ready to fire configuration is about 2 to about 8 pounds of pressure.

20. The drug delivery device of claim 1, wherein the force required by a user to activate the firing mechanism is about 2 to about 8 pounds of pressure.

21. The drug delivery device of claim 1, wherein the mechanically advantaged mechanism is a lever or an inclined plane.

22. A ophthalmic drug delivery device comprising:
a housing configured to contain one or more ampoules comprising an ophthalmic drug in a compartment, wherein the ampoules comprise a piercable region in the compartment;
a firing mechanism effective to apply pressure to an ampoule effective to cause the ampoule to be pierced at the piercable region and to release the drug under pressure through the piercable region of the compartment;
a delivery device configured to direct the released drug to the eye of a user;
a mechanically advantaged mechanism whereby a user positions the device into a ready to fire configuration; and
a mechanically advantaged firing control whereby a user activates the firing mechanism to release the drug from the ampoule.

23. The drug delivery device of claim 21, wherein the drug is preservative-free.

24. The drug delivery device of claim 21, wherein the drug is sterile until released from the ampoule.

25. The drug delivery device of claim 21, wherein the ampoule is a single-dose ampoule or a two-dose ampoule.

26. The drug delivery device of claim 22, wherein the ampoule is selected from the group consisting of internally pierced ampoules, cutaway tip ampoules, externally pierced ampoules, burst ampoules, and self-piercing ampoules.

27. The drug delivery device of claim 22, wherein the ampoule is pierced externally.

28. The drug delivery device of claim 22, wherein the ampoule is pierced internally.

29. The drug delivery device of claim 22, wherein the ampoule further comprises a head space of gas or air.

30. The drug delivery device of claim 22, wherein the mechanically advantaged mechanism is a lever or an inclined plane.

31. The drug delivery device of claim 22, wherein the delivery device is designed for a single user.

32. The drug delivery device of claim 31, wherein the delivery device has a clamshell, disk, or cylindrical configuration.

33. The drug delivery device of claim 22, wherein the delivery device is designed for institutional administration.

34. The drug delivery device of claim 33, wherein the delivery device has a revolver configuration.

35. The drug delivery device of claim 22, wherein the delivery device further comprises an eye cup.

36. The drug delivery device of claim 35, wherein the eye cup in fluid communication with the piercable region and directs the release of the drug to the eye of a user.

37. The drug delivery device of claim 35, wherein the eye cup comprises an eye guide.

38. The drug delivery device of claim 35, wherein the eye cup is an Integrated Ampoule Ophthalmic Dispenser.

39. The drug delivery device of claim 35, wherein the eye cup is collapsible.

40. The drug delivery device of claim 35, wherein the eye cup comprises notched cutouts on one or more sides of the eye cup.

41. The drug delivery device of claim 22, wherein the ampoules are in an Ampoule Cartridge Holder.

42. The drug delivery device of claim 41, wherein the Ampoule Cartridge Holder comprises an indexing orientation key.

43. The drug delivery device of claim 41, wherein the Ampoule Cartridge Holder comprises one or more aids for administering the ampoules to a user selected from the group consisting of numbering, color coding, icon system coding, Braille, bar coding, and Radio Frequency Identification Device.

44. The drug delivery device of claim 22, wherein the device further comprises a programmable microprocessor.

45. The drug delivery device of claim 44, wherein the programmable microprocessor is a Printed Circuit Board or an Application Specific Integrated Circuit coupled to visual display interface.

46. The drug delivery device of claim 45, wherein the visual display interface is a Liquid Crystal Display or Light Emitting Diodes.

47. The drug delivery device of claim 22, wherein the drug is released from the ampoule into the eye of a user approximately 200 to 300 milliseconds after the user activates the firing mechanism.

48. An intranasal drug delivery device comprising:
a housing configured to contain one or more ampoules comprising a substance adapted for intranasal delivery in a compartment, wherein the ampoules comprise a piercable region in the compartment;
a firing mechanism effective to apply pressure to an ampoule effective to cause the ampoule to be pierced at the piercable region and to release the substance under pressure through the piercable region of the compartment;
a nozzle configured to direct the released substance to the nasal passage of a user;
a mechanically advantaged mechanism whereby a user positions the device into a ready to fire configuration; and
a mechanically advantaged firing control whereby a user activates the firing mechanism to release the substance from the ampoule.

49. The drug delivery device of claim 48, wherein the substance is sterile until released from the ampoule.

50. The drug delivery device of claim 48, wherein the ampoule is a single-dose ampoule or a two-dose ampoule.

51. The drug delivery device of claim 48, wherein the ampoule is selected from the group consisting of internally pierced ampoules, cutaway tip ampoules, externally pierced ampoules, burst ampoules, and self-piercing ampoules.

52. The drug delivery device of claim 48, wherein the firing mechanism is a plunger.

53. The drug delivery device of claim 52, wherein the plunger comprises a geometrically shaped ridge guide.

54. The drug delivery device of claim 48, wherein the nozzle is an Integrated Ampoule Nozzle Dispenser.

55. The drug delivery device of claim 48, wherein the nozzle angles the release of the substance from the ampoule 20 to 80 degrees.
